SEQUENCE LISTING (6 sequences)

SEQ ID NO: 1 - DNA, 1610 bp, HIV
  DNA coding sequence for HIV capsid protein Pr55
atgggtgcgagagcgtcggtattaagcgggggagaattagataaatgggaaaaaattcggttaaggccagggggaaagaaaaaatataagttaaaacatatagtatgggcaagcagggagctagaacgattcgcagtcaatcctggcctgttagaaacatcagaaggctgcagacaaatattgggacagctacagccatcccttcagacaggatcagaagaacttagatcattatataatacagtagcaaccctctattgtgtacatcaaaggatagatgtaaaagacaccaaggaagctttagagaagatagaggaagagcaaaacaaaagtaagaaaaaggcacagcaagcagcagctgcagctggcacaggaaacagcagccaggtcagccaaaattaccctatagtgcagaacctacaggggcaaatggtacatcaggccatatcacctagaactttaaatgcatgggtaaaagtagtagaagaaaaggctttcagcccagaagtaatacccatgttttcagcattatcagaaggagccaccccacaagatttaaacaccatgctaaacacagtggggggacatcaagcagccatgcaaatgttaaaagagactatcaatgaggaagctgcagaatgggatagagtgcatccagtgcatgcagggcctattgcaccaggccaaatgagagaaccaaggggaagtgacatagcaggaactactagtacccttcaggaacaaataggatggatgacaaataatccacctatcccagtaggagaaatctataaaagatggataatcctgggattaaataaaatagtaagaatgtatagccctaccagcattctggacataagacaaggaccaaaggaaccctttagagattatgtagaccggttctataaaactctaagagccgaacaagcttcacaggatgtaaaaaattggatgacagaaaccttgttggtccaaaatgcaaacccagattgtaagactattttaaaagcattgggaccagcagctacactagaagaaatgatgacagcatgtcagggagtggggggacccggccataaagcaagagttttggctgaagccatgagccaagtaacaaatccagctaacataatgatgcagagaggcaattttaggaaccaaagaaagactgttaagtgtttcaattgtggcaaagaagggcacatagccaaaaattgcagggcccctaggaaaaagggctgttggagatgtggaagggaaggacaccaaatgaaagattgcactgagagacaggctaattttttagggaagatctggccttcctacaagggaaggccagggaattttcttcagagcagaccagagccaacagccccaccagaagagagcttcaggtttggggaggagaaaacaactccctctcagaagcaggagccgatagacaaggaactgtatcctttaacttccctcagatcactctttggcaacgacccctcgtcacaataaggataggggggcaactaaaggaagctctattagatacaggagcagatgatacagtattagaagaaatgaatttgccaggaaaatggaaaccaaaaatgata

SEQ ID NO: 2 - protein, 24 aa, Triticum aestivum
  peptide fragment of host cell (wheat germ) protein HP68
PRPYLDVKQRLKAARVIRSLLRSN

SEQ ID NO: 3 - DNA, 44 bp, Artificial Sequence
  Degenerate oligonucleotide C-terminal peptide sequence of WGHP68
atgaattcactgggactgcggatagattactggtactggggatc

SEQ ID NO: 4 - DNA, 42 bp, Artificial Sequence
  Degenerate oligonucleotide C-terminal peptide sequence of WGHP68
atgaattcactgggctctgatagattactggtactggggatc

SEQ ID NO: 5 - protein, 604 aa, Triticum aestivum
MADRLTRIAIVSEDKCKPKKCRQECKKSCPVVKTGKLCIEVSPVAKLAFISEELCIGCGICVKKCPFDAIEIINLPKDLEKDTTHRYGPNTFKLHRLPVPRPGQVLGLVGTNGIGKSTALKVLAGKLKPNLGRFKNPPDWQEILTYFRGSELQNYFTRILEDNLKAIIKPQYVDHIPKAVQGNVGQVLEQKDERDMKNELCVDLELNQVIDRNVGDLSGGELQRFAIAVVAVQSAEIYMFDEPSSYLDVKQRLKAARVIRSLLRSNSYVIVVEHDLSVLDYLSDFICCLYGKPGAYGVVTLPFSVREGINIFLAGFVPTENLRFRDESLTFKIAETQESAEEVATYQRYKYPTMSKTQGNFKLSVVEGEFTDSQIVVMLGENGTGKTTFIRMLAGLLKPDTMEGTEVEIPEFNVSYKPQKISPKFQHPVRHLLHSKIRDSYTHPQFVSDVMKPLQIEQLMDQEVINLSGGELQRVALCLCLGKPADIYLIDEPSAYLDSEQRIVASKVIKRFILHAKKTAFIVEHDFIMATYLADKVIVYEGLASIDCTANAPQSLVSGMNKFLSHLDITFRRDPTNYRPRINKLESTKDREQKNAGSYYYLDD

SEQ ID NO: 6 - protein, 599 aa, Homo sapiens
MADKLTRIAIVNHDKCKPKKCRQECKKSCPVVRMGKLCIEVTPQSKIAWISETLCIGCGICIKKCPFGALSIVNLPSNLEKETTHRYCANAFKLHRLPIPRPGEVLGLVGTNGIGKSAALKILAGKQKPNLGKYDDPPDWQEILTYFRGSELQNYFTKILEDDLKAIIKPQYVARFLRLAKGTVGSILDRKDETKTQAIVCQQLDLTHLKERNVEDLSGGELQRFACAVVCIQKADIFMFDEPSSYLDVKQRLKAAITIRSLINPDRYIIVVEHDLSVLDYLSDFICCLYGVPSAYGVVTMPFSVREGINIFLDGYVPTENLRFRDASLVFKVAETANEEEVKKMCMYKYPGMKKKMGEFELAIVAGEFTDSEIMVMLGENGTGKTTFIRMLAGRLKPDEGGEVPVLNVSYKPQKISPKSTGSVRQLLHEKIRDAYTHPQFVTDVMKPLQIENIIDQEVQTLSGGELQRVRLRLCLGKPADVYLIDEPSAYLDSEQRLMAARVVKRFILHAKKTAFVVEHDFIMATYLADRVIVFDGVPSKNTVANSPQTLLAGMNKFLSQLEITFRRDPNNYRPRINKLNSIKDVEQKKSGNYFFLDD

(19) United States
(12) Patent Application Publication
(10) Pub. No.: US 2003/0104577 A1
(43) Pub. Date: Jun. 5, 2003

(54) HIV capsid assembly-associated compositions and method

(75) Inventors: Vishwanath R. Lingappa, San Francisco, CA (US); Jaisri R. Lingappa, Seattle, WA (US)

Correspondence Address: BARBARA RAE-VENTER, RAE-VENTER LAW GROUP, P.C., PO BOX 60039, PALO ALTO, CA, 94306, US

(73) Assignee: UC Regents

(21) Appl. No.: 10/040,206

(22) Filed: Jan. 2, 2002

Related U.S. Application Data
(63) Continuation-in-part of application No. 09/020,144, filed on Feb. 6, 1998.
(60) Provisional application No. 60/039,309, filed on Feb. 7, 1997.

(51) Int. Cl. C07K 14/16; C12P 21/02; C12N 5/08; C12N 15/867

(52) U.S. Cl. 435/69.3; 435/456; 530/350; 435/366; 435/320.1

(57) ABSTRACT

A cell-free method for translation and assembly of retroviral, particularly HIV, capsid and capsid intermediates is disclosed. Also disclosed are novel HIV capsid assembly intermediates and novel host proteins which bind to such assembly intermediates. The invention also includes a screening method for compounds that alter retrovirus capsid assembly, and a method of treating HIV using compounds which inhibit the HIV capsid assembly pathway.

### CROSS-REFERENCE TO RELATED APPLICATIONS

This application claims benefit of U.S. Ser. No. 60/039,309 filed Feb. 7, 1997 and U.S. Ser. No. 09/020,144, filed Feb. 6, 1998, which disclosures are hereby incorporated by reference.

### STATEMENT REGRADING FEDERALLY SPONSORED RESEARCH

This invention was made with Government support by Grant Nos. K08AI01292 and A141881, awarded by the National Institutes of Health (NIH) and National Institutes of Health AIDS Division, respectively. The Government has certain rights in this invention.

### FIELD OF THE INVENTION

The invention is concerned with a method for producing HIV capsids in a cell-free extract. Also described are capsid intermediate compositions, auxiliary proteins, and screening assays that measure the ability of drugs to inhibit this process.

### BACKGROUND OF THE INVENTION

The protein shell of the HIV virion, termed the HIV capsid or core, is composed of approximately 1500 copies of the Pr55 Gag structural protein precursor (Gelderblom, H. R., AIDS 5:617-638 (1991)). For proper assembly of the capsid to occur, Pr55 chains must undergo myristoylation (Gheysen, D. et al., Cell 59:103-112 (1989); Gottlinger, H. G., et al., Proc. Natl. Acad. Sci. 86:5781-5785 (1989)), an N-terminal modification thought to occur co-translationally (Towler, D. A., et al., Ann. Rev. Biochem. 57:69-99 (1988)). The myristoylated chains are targeted to the host plasma membrane where assembly takes place concomitant with RNA encapsidation. As capsids are formed, they bud into the plasma membrane. This results in envelopment and subsequent release of viral particles from the cell. Coincident with their release, the immature viral particles undergo a maturation process, involving proteolytic processing of the precursor structural proteins and condensation of the capsids into collapsed, electron-dense cores (Gelderblom, H. R., AIDS 5:617-638 (1991); Wills, J. W. and Craven, R. C., AIDS 5:639-654 (1991)).

The manner in which HIV capsids assemble differs from that of many other retroviruses. Other retroviruses of the type B and type D category assemble “preformed” capsids in the cytoplasm of the infected cells. Such preformed capsids are then transported to other areas of the cell, such as the plasma membrane. In contrast, HIV capsids and other type C retroviruses form in intimate association with the plasma membrane, as described above. This important characteristic of HIV capsid formation has been demonstrated through electron microscopic studies (reviewed by Gelderblom, H. R., AIDS 5:617-638 (1991 Wills, J. W. and Craven, R. C., AIDS 5:639-654 (1991)).

Analyses of various mutants of Pr55 have revealed key domains required for efficient capsid assembly and targeting to the plasma membrane (see for Gheysen, D. et al., Cell 59:103-112 (1989); Gottlinger, H. G., et al., Proc. Natl. Acad. Sci. 86:5781-5785 (1989); Trono, D., et al., Cell 59:113-120 (1989); Royer, M., et al., Virology 184:417-422 (1991); Jowett, J. B. M., et al., J. Gen. Virol. 73:3079-3086 (1992); Facke, M. et al., J. Virol 67:4972-4980 (1993); Wang, C.-T. and Barklis, E., J. Virol. 67:4264-4273 (1993); Spearman, P. et al., J. Virol. 68:3232-3242 (1994); Hockley, D. J. et al., J. Gen. Virol. 75:2985-2997 (1994); Zhao, Y., et al., Virology 199:403-408 (1994)). However, the actual mechanisms involved in coordinating the formation of an HIV capsid from 1500 Gag monomers have not been elucidated. Many important questions about HIV capsid assembly remain unanswered, including whether assembly is an energy-dependent process, whether host proteins are required for assembly to take place, and whether assembly proceeds by way of discrete intermediates.

A major obstacle to addressing these questions experimentally has been the inherent difficulty of studying capsid assembly in cellular systems. In cells, many of the events in question proceed extremely rapidly and are not readily amenable to manipulation, making it difficult to identify trans-acting factors and energy substrates that may be required for assembly.

An important aspect of understanding the HIV life cycle, including capsid assembly, is the ability to develop anti-HIV drugs that effectively abolish replication. The anti-HIV drugs currently being used to treat patients infected with HIV either have minimal anti-HIV activity, produce adverse side effects or both. 3′-azido-3′-deoxythymidine (Zidovudine, AZT), the most widely recommended and used anti-HIV drug, has recently been shown to be ineffective in blocking HIV replication as a reverse transcriptase inhibitor (Papadopulos-Eleopulos et al. Curr Med Res Opin. (1991) Suppl. 1:S1-45). The triphosphorylated form of the drug does posses anti-HIV properties, however the unphosphorylated form is administered to patients and this form is not phosphorylated in vivo. In addition AZT has many adverse side effects, which in combination with its inability to reduce viral load provide for a very ineffective treatment option for HIV infected patients.

The highly active anti-retroviral therapy (HAART) is also widely used but after a few years of therapy, where no detectable viral load can be measured, it has been shown that when patients are taken off the therapy, relapse occurs in almost all individuals (Smith K. Curr Opin Immunol. (2001) 13(5):614-24). HAART prolongs the life of chronically infected patients, however extended, if not indefinite, use of the drugs is required. With time, the use of HAART has adverse side effects, such as lactic acidosis, lipodystrophy (fat redistrubution, hyperlipidemia), diabetes mellitus and the promotion of drug resistant strains of HIV (Isada C. Cleve Clin J. Med (2001) 68(9):804-7; Jain et al. Antiviral Res. (2001) 51(3):151-77). Other anti-HIV drugs include protease inhibitors such as saquinavir, ritonavir, indinavir, nelfinavir and amprenavir (Ren et al. Prog Drug Res. (2001) Spec No: 1-34). These drugs are ineffective at completely abolishing HIV replication.

It therefore is of interest to identify the individual steps involved in HIV immature capsid assembly and to determine both the intermediates and the identity and conformation of trans acting host proteins involved in the HIV immature capsid assembly cascade as a means of developing compounds which inhibit not the host proteins identified but the specific conformers of the host proteins that are involved in the HIV capsid assembly cascade. There is a need for compounds for treatment of HIV infected individuals that specifically inhibit HIV replication, but do not have significant side effects and do not promote new strains of HIV that are resistant to treatment.

### SUMMARY OF THE INVENTION

This invention relates to methods for isolating Gag intermediates in the assembly cascade for immature HIV capsids, identifying host protein conformers that bind to these intermediates, using the conformers to develop treatments for HIV that specifically target the identified conformers and not other conformers of these proteins, and making conformer specific antibodies together with compositions that include the identified conformers, the HIV capsid assembly cascade intermediates, conformer-intermediate complexes and antibodies to the conformers and the conformer-intermediate complexes. The method for isolating the intermediates includes the steps of adding HIV Gag Pr55 mRNA to a cell-free protein translation mixture supplemented with myristoyl coenzyme A and optionally one or both of a detergent sensitive and a detergent insensitive fraction derived from eukaryotic cell membranes; incubating the resulting mixture for a time sufficient to assemble Gag Pr55 mRNA translation products into an immature HIV capsid; separating the intermediate-host protein complexes that have formed; and isolating the complexes. The HIV capsid assembly intermediates isolated include Gag-containing intermediates having buoyant densities of about 10 S, about 80 S, about 150 S and about 500 S. These assembly intermediates can be used as components in a screening assay for conformers of host proteins involved in capsid assembly as well as in a screening assay for compounds which specifically inhibit the trans-acting host proteins. Such compounds would block the intermediates from assembling into HIV capsids. The intermediates and the host protein complexes can be affinity purified using anti-Gag antibodies and the intermediate and the associated host protein conformer separated. The host protein conformer can then be sequenced and the sequence used to identify other protein conformers i.e. proteins with substantially the same amino acid sequence as the host protein conformer but which do not bind to any of the Gag containing intermediates and do not promote immature HIV capsid assembly when added to a cell-free protein translation mixture depleted of the host protein conformer.

Monoclonal antibodies specific for each of the host protein conformer and the other protein conformers identified can be made by immunizing knock out animals, which lack a functional gene for the host protein and do not produce the protein, with one of the conformers; preparing hybridomas from the spleens of the immunized animals; and screening the monoclonal antibodies produced for antibodies that bind substantially specifically to only one conformer. Identification of compounds that will interfere with binding of the host protein conformers to particular Gag intermediates includes epitope mapping the binding site on the host protein conformer for the Gag intermediate; screening databases for compounds that will bind to the identified binding site; and selecting from among the compounds identified those that bind to the host protein conformer and do not bind to other conformers of the protein. The invention finds use in identifying compounds that specifically affect the function of host protein conformers that are trans-acting factors involved in HIV capsid formation and which can be expected to specifically inhibit HIV capsid formation without affecting the functions associated with other conformers of the protein. It also finds use in the development of conformer profiles that can be used in patient prognosis and in identifying optimal treatment regimens for individual patients with HIV.

### BRIEF DESCRIPTION OF THE FIGURES

FIG. 1 shows migration of capsids formed in a cell free system (FIG. 1A) and in a cellular system (FIG. 1B) on velocity sedimentation gradients, in the form of plots of the buoyant density of each of the sequential fractions collected, assessed by refractive index (open circles), and of the amount of Gag protein in each fraction, as assessed by densitometry (closed circles).

FIG. 2 shows the amount of capsid assembly occurring in a cell free system in the presence of MCoA added at different time points during the reaction (FIG. 2A) and in the presence of two different concentrations of “NIKKOL” (FIG. 2B).

FIG. 3 shows bar graphs demonstrating the effect on assembly of inhibition of protein synthesis and depletion of ATP fifty minutes into the reaction (FIG. 3A) and the requirement for a membrane fraction in the reaction (FIG. 3B)

FIG. 4 shows schematic diagrams of mutations within Gag (FIG. 4A), and the amount (FIG. 4B) of capsid assembly that occurred in the cell-free system primed with transcripts of the various mutant HIV viruses shown in FIG. 4A, as well as wild-type capsids (WT) and capsids produced in the absence of MCoA (-MCoA).

FIG. 5 shows pulse-chase analysis of HIV capsid assembly by velocity sedimentation in a continuously labeled cell-free reaction mixture (FIG. 5A) where the calculated positions of 10S, 80S, 150S, 500S, and 750S complexes are indicated by markers at the top of the graph, and in reactions to which unlabeled ³⁵S cysteine was added 4 minutes into the reaction and aliquots were taken for sedimentation analysis after 25 minutes (FIG. 5B) and 15 minutes of reaction (FIG. 5C), and samples were further analyzed by SDS gel and radiography.

FIG. 6 shows plots of pulse-chase experiments in which transcripts of different assembly-defective mutants Pr46 (FIG. 6B), Pr41(FIG. 6C), GΔA (FIG. 6D), and D2 (FIG. 6E) and wild-type HIV (WT; FIG. 6A) were analyzed for assembly in a cell-free system.

FIG. 7 shows plots of sedimentation of Gag complexes isolated from COS-1 cells transfected with a transfection vector encoding Pr55 cDNA wild-type Gag (FIG. 7A) or by transfection vectors encoding the p41 mutant (FIG. 7B) or the D2 mutant (FIG. 7C).

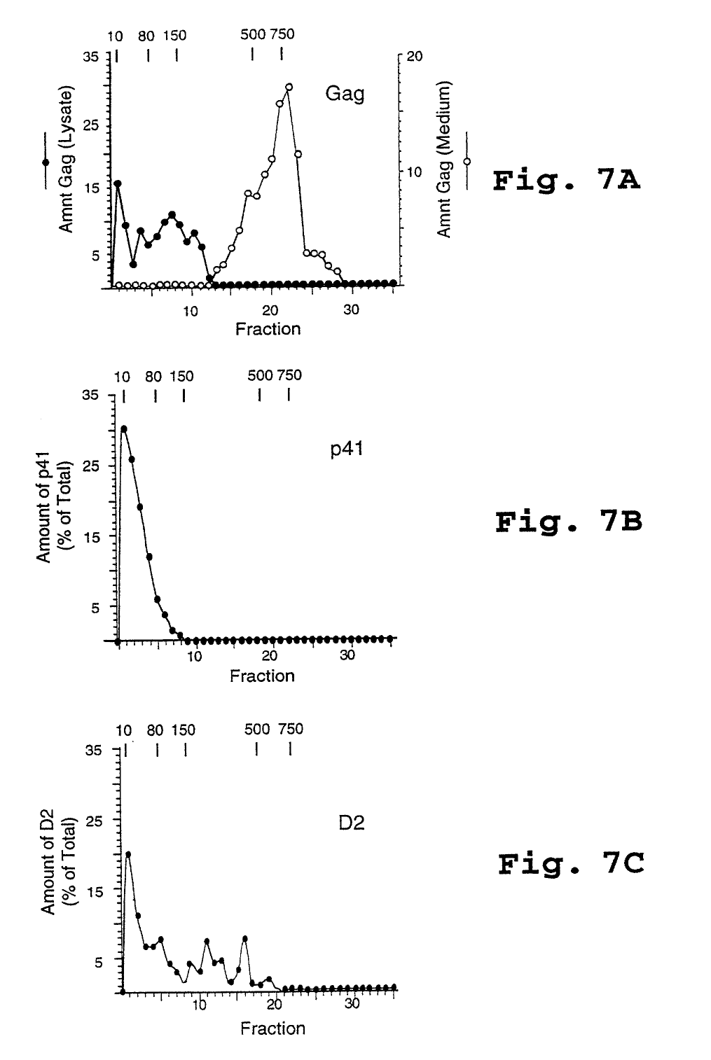

FIG. 8 shows a schematic model for assembly of immature HIV capsids (FIG. 8A) and the points along the pathway at which Gag mutants p41 (FIG. 8B), GΔA or wild-type in the absence of MCoA (Wt−MCoA; FIG. 8C), D2 (FIG. 8D) are arrested, compared to wild type in the presence of MCoA (WT+MCoA) or p46 (FIG. 8E).

FIG. 9 shows alignment of WGHP68 (SEQ ID NO: 5) with HuHP68 (SEQ ID NO: 6). Dashes indicate alignment gaps; asterisks, identical amino acids; dots, conserved amino acids. Open boxes; P-loop motifs. Black boxes; regions sequenced and used for constructing degenerate oligonucleotides. Arrows: residue before stop codon in WGHP68-Tr1.

FIG. 10 shows HuHP68 co-immunoprecipitates HIV-1 Gag in mammalian cells. Native (NATIVE) or denaturing (DENAT) immunoprecipitations using αHuHP68b (HP) or non-immune serum (N), followed by immunoblotting (IB) with antibody to HuHP68 (IB: HP) or Gag (IB: Gag), were performed on: (FIG. 10A) 293T cells transfected with pBRUΔenv, +/− RNase A treatment; (FIG. 10B), Cos-1 cells expressing Gag; (FIG. 10C), Cos-1 cells expressing Gag (Gag), an assembly-incompetent Gag mutant (p41), an assembly-competent Gag mutant (p46), or control vector (native immunoprecipitation only); or (FIG. 10D), chronically HIV-1-infected ACH-2 cells. HIV-1 p24 and p55 (arrows), 5% input cell lysate (T), and 10 μl medium (T medium) are indicated.

FIG. 11 shows co-localization of HP68 with HIV-1 Gag in mammalian cells. (FIGS. 11A-I), Cos-1 cells were transfected with pBRUΔenv or pBRUp41Δenv (truncated proximal to the nucleocapsid domain in Gag), and double-label indirect immunofluorescence was performed. Fields were labeled for HP68 (red, top row: FIGS. 11A, D, G), or Gag (green, middle row: FIGS. 11B, E, H). Images were merged to show overlap of HP68 and Gag labeling (yellow; bottom row: FIGS. 11C, F, I). Bar at lower right corresponds to 50 μM.

FIG. 12 shows truncated HP68 blocks virion production. (FIGS. 12A-D), Cos-1 (FIGS. 12A, B) or 293T (FIGS. 12C, B) cells co-transfected with varying amounts of plasmid expressing WGHP68-Tr1 and empty vector, as indicated, plus plasmids for expression of HIV-1 Gag (FIGS. 12A, B) or pBRUΔenv (FIGS. 12C, B). Medium (FIGS. 12A, C) was immunoblotted with Gag antibody (p55; p24), and reprobed with antibody to light chain tracer (LC). Cell lysates (FIGS. 12B, D) were immunoblotted using WGHP68 antiserum (HP) or Gag antibody (p55; p24), and reprobed using actin antibody (actin). Arrows: open, native HP68; filled, WGHP68-Trl. Bar graphs: blots from 3 experiments quantitated using sample dilution standard curves.

FIG. 13 shows HP68 depletion-reconstitution. (FIGS. 13A-B), Graphs show total Gag synthesized (FIG. 13A) or amount of Gag in 750S completed capsids (FIGS. 13B) from cell-free reactions programmed with indicated WG extracts: non-depleted; immunodepleted (depleted); or immunodepleted reconstituted with either GST alone (+GST), WGHP68-GST (+WGHP68), or HuHP68-GST (+HuHP68). (FIG. 13C), Amount of Gag in fractions from cell-free reactions in A that were subjected to velocity sedimentation. (FIGS. 13D, E), TEM of capsids from immunodepleted cell-free reactions reconstituted with WGHP68-GST (13D) or immature capsids from transfected mammalian cells (FIG. 13E). Bar: 100 nm. (FIG. 13F), Proteinase K digestion of 500S and 750S fractions shown as % Gag protected relative to normalized controls. Open circle in 13D is depleted and closed circles are reconstituted (GST); filled bars in 13E are 500S intermediates and diagonal lines are 750S assembled capsids

FIG. 14 shows HuHP68 co-immunoprecipitates HIV-1 Gag and Vif but not Nef or RNase L. (FIG. 14A), Cos-1 cells transfected with pBRUΔenv or HIV-1 Gag plasmids were immunoprecipitated under native (NATIVE) or denaturing (DENAT) conditions using αHuHP68b (HP) or non-immune serum (N), and immunoblotted (IB) with antibody to HuHP68 (HP), HIV-1 Gag, HIV-1 Vif, HIV-1 Nef, RNase L (RL), or Actin. Total (T): 5% of input cell lysate used in immunoprecipitation (HP: 10%). Top of some actin lanes contains heavy chain cross-reacting to secondary. (FIG. 14B) shows the results with lysates of pERUΔenv-transfected Cos-1 cells, harvested in 10 mM EDTA-containing buffer, and co-immunoprecipitated using beads pre-incubated with HuHP68 peptide or diluent control.

### DETAILED DESCRIPTION OF THE INVENTION

The present invention uses a cell-free system for translation and assembly of HIV capsids as a means of identifying capsid assembly intermediates and trans acting host proteins involved in capsid assembly. This information is then used in the identification of antiviral compounds that inhibit the conformers of host proteins that are involved in viral replication; candidate compounds also can be screened using the cell free translation system. The phrase “cell-free translation” refers to protein synthesis carried out in vitro in a cell extract that is essentially free of whole cells. The phrase “cell-free translation mixture” refers to a cell free extract that generally includes sufficient cellular machinery and components to support protein translation which include transfer RNA, ribosomes, a full complement of at least 20 different amino acids, an energy source, which may be ATP and/or GTP, and an energy regenerating system, such as creatine phosphate and creatine phosphokinase. The term “conformer” refers to a protein having at least substantially the same amino acid sequence, but heterogeneity in structure (physical topology or topography) and function. By topology is intended the different placement of the protein, e.g. C-cytosolic as compared to N-cytosolic, and topography intends change in external conformation or shape, (i.e different three-dimensional shape due to differences in folding/conformation), which includes stable and transient association with other proteins. As used herein, polypeptides of substantially the same amino acid sequence are those with conservative amino acid substitutions (i.e. a small or large side chain for a small or large side chain, respectively; or an acidic, basic, polar or hydrophobic side chain for an acidic, basic, polar or hydrophobic side chain, respectively), that do not alter the protein conformation or topology. The protein conformation changes are due to post-translational modifications and are not a factor of the amino acid sequence.

The cell-free system is programmed with a mRNA molecule encoding HIV Pr55 Gag protein or a plasmid encoding the Gag protein, immature HIV capsids are produced after incubation for a period of time sufficient to assemble Gag Pr55 mRNA translation products.

The subject invention offers several advantages over existing technology. The cell-free system that recreates capsid biogenesis greatly facilitates a biochemical dissection and mechanistic understanding of capsid formation. Immature HIV capsids can be assembled in a cell-free protein translation system, when certain key components are added to the reaction. Capsid formation by this method has the same requirement as capsid formation in vivo, including a requirement for myristoylation of Gag and an apparent requirement for membranes. Furthermore, this method for cell-free assembly of HIV capsids reveals the existence of previously unknown steps in HIV virus formation, allowing disassociation of the process of capsid formation into co- and post-translational phases, each of which has distinct co-factor and/or energy requirements. Using such a system, the post-translational phase can be shown to be dependent on ATP and at least two independent host factors, which are distinguished by their differential sensitivities to non-ionic detergents.

Another advantage of the subject invention is finding that formation of HIV capsids proceeds by way of a pathway of previously unrecognized assembly intermediates, in both cells and in the cell-free system. These previously unknown intermediates can be used in the design of drugs (including peptides and antibodies) and vaccines that interfere with progression from one intermediate to the next, in the design of drugs that act by inhibiting host cell machinery involved in capsid formation, and in the design of assay systems that examine the efficacy and mechanism of action of drugs that inhibit capsid formation.

This system also offers the advantage that it can be used for identifying drugs that interfere with the process of capsid formation. Such a system would include a screening assay for host protein conformers functioning as chaperones in viral replication or as a selection assay for identification of new compounds that interfere with capsid formation by specifically inhibiting the chaperone conformers, and hence with production of infectious virus. An exemplary host protein, termed HP68, is a 68 kD protein present in a cell-free fraction of wheat germ extract and which forms part of one or more of the intermediate complexes described above. Not only is this protein useful as a component of the cell-free translation systems and methods described above, but it can be used to design drugs that block or alter its association with HIV Gag and Vif and which therefore prevent formation of immature HIV capsids. A particularly important advantage is the identification of a conformational difference between HP68, which possesses activity that inhibits RNAse L, a cellular protein that promotes degradation of viral RNA, and indirectly functions to facilitate viral replication and the conformer of HP68 that directly promotes viral replication by binding Gag during virion formation. By targeting the latter conformer as opposed to the one that acts indirectly to facilitate viral replication, HIV replication can be specifically inhibited without undesirable side effects. Furthermore, since the target for the drug is a host protein rather than a viral protein, there is a decreased likelihood of the development of viral resistance to such a drug.

Another advantage of the subject invention is the discovery that pieces of genomic HIV RNA can be encapsidated into the HIV capsids produced in the cell-free system by adding such RNA to the system. This feature of the invention can be used to design drugs that interfere with encapsidation and in the design of assay systems that examine the mechanism of actions of drugs that inhibit encapsidation.

The present invention includes a method for producing HIV capsid assembly intermediates in a cell-free system. Assembly of immature capsids in cells requires expression of only the HIV Pr55 protein, however standard in vitro translation systems, that include a cytosolic extract, amino acids, an ATP regenerating system, and in vitro synthesized transcript coding for Pr55 Gag fail to support assembly of HIV capsids. It was found necessary to add sufficient myristoyl coenzyme A (MCoA) to the system to enable assembly of HIV capsids. The term “assembly intermediates” refers to capsid substructures (composed of Gag polypeptides as well as other, as-yet-undefined components) that must be formed in an ordered sequence in order for the final completed capsid structure to be made. The term “assembly pathway” refers to the ordered set of serial assembly intermediates required for formation of the final completed capsid structure. To progress from one assembly intermediate to the next, a specific modification or modifications of the intermediate must take place. These modifications are not completely defined and are likely to include addition of more Gag polypeptides, host-mediated modifications of the intermediate, and association with host factors as exemplified by HP68.

Known in the art are a number of in vitro translated systems, the basic requirements of which have been well-studied (Erickson and Blobel, Methods Enzymol (1983) 96:38-50; Merrick, W. C., Methods Enzymol. (1983) 101:606-615; Spirin et al. Science (1988) 242:1162-1164). Examples include wheat germ extract and rabbit reticulocyte extract, available from commercial suppliers such as Promega (Madison, Wis. ), as well as high speed supernatants formed from such extracts. While the cell-free translation mixture can be derived from any of a number of cell types known in the art, the cell-free system of the present invention is exemplified using wheat germ cell-free extract. This cell-free translation mixture of wheat germ extract can be programmed with HIV genomic RNA or a fraction thereof, whereby the system is capable of making capsids containing HIV RNA. The term “programmed with” means addition to a cell-free translation mixture or cells, mRNA that encodes HIV proteins, or by adding to cells a DNA sequence that specifies the production of such HIV protein. Viral mRNA can be added to cells directly, such as by transfection or electroporation according to methods well known in the art. DNA that directs the production of mRNA can also be used to program the cell-free system or “added to cells” by inserting the corresponding gene into an appropriate vector and transfecting the cell. This system can also be supplemented with exogenous proteins, such as HP68 which facilitate the assembly of HIV capsid intermediates. To produce mutant capsid intermediates, the system is programmed with HIV mutants, such as Pr46, Pr41, GΔA and D2, which are well known in the art.

Methods known in the art are used to maintain energy levels sufficient to maintain protein synthesis, for example, by adding additional nucleotide energy sources during the reaction or by addition of an energy source, such as creatine phosphate/creatine phosphokinase. The ATP and GTP concentrations present in the standard translation mixture, generally between about 0.1 and 10 mM, more preferably between about 0.5 and 2 mM, are sufficient to support both protein synthesis and capsid formation, which may require additional energy input. Generally, the reaction mixture prepared in accordance with the present invention, as exemplified in Example 1, can be titered with a sufficient amount of ATP and/or GTP to support production of a concentration of about 10 picomolar Gag in the system. The translation mixture may also include the detergent-sensitive, detergent-insensitive, and host protein fractions described below, or it may be supplemented with such fractions. The term “detergent-sensitive fraction” refers to a component most likely containing a membrane lipid bilayer that is present in a standard wheat germ extract prepared according to the methods described by Erickson and Blobel (1983), which component is deactivated with reference to supporting HIV capsid assembly when a concentration of 0.1% (wt/vol) “NIKKOL” is added to the extract. It is appreciated that such a detergent-sensitive factor can be present in extracts of other cells similarly prepared, or can be prepared independently from a separate cell extract, and then added to a cell-free translation system.

The cell-free translation reaction is initiated by adding HIV Gag Pr55 mRNA, the sequence of which is known in the art, or can be derived from the DNA sequence provided herein as SEQ ID NO: 1 to the cell free translation mixture. Suitable mRNA preparations include a capped RNA transcript produced in vitro using the mMESSAGE mMACHINE kit (Albion). MRNA molecules can also be generated in the same reaction vessel as is used for the translation reaction by addition of SP6 or T7 polymerase to the reaction mixture, along with the HIV Gag coding region or cDNA. This coding region encoding Gag Pr55 can be obtained, for example, by DNA synthesis according to standard methods, using the sequence provided as SEQ ID NO: 1. Alternatively the plasmid described in Example 6 (FIG. 16), pBRUΔenv, which codes the entire HIV genome except for the envelope protein sequence, can be used.

The cell-free translation mixture is supplemented with myristoyl coenzyme A in an amount sufficient to support capsid formation. While the concentration required will vary according to the particular experimental conditions, in experiments carried out in support of the present invention, it was found that a concentration between about 0.1 and 100 μM and preferably between about 5 and 30 μM supports HIV capsid formation. Without committing to a particular theory concerning the mechanism of the reaction, it is likely that this supplement promotes myristoylation of the Gag translation product and attachment to membrane fragment(s) present in the cell free translation mixture. When the membranes present in the cell free translation mixture are solubalized by addition of detergent, it is shown that assembly of the HIV capsid is sensitive to addition of detergent above but not below the critical micelle concentration. This observation is consistent with a role for membranes being required at a particular step in capsid assembly. Furthermore, HIV capsid assembly is improved by the presence of a cellular component that has a sedimentation value greater than 90 S in a sucrose gradient and is insensitive to extraction with at least 0. 5% “NIKKOL”.

The cell-free capsid assembly reaction described above can be extended to include packaging of RNA, by addition of genomic HIV RNA or fragments thereof during the capsid assembly reaction. Addition and monitoring of RNA encapsidation provides an additional parameter of HIV particle formation that can be exploited in drug screening assays, in accordance with the present invention.

The HIV RNA sequence to be used for making the HIV genomic RNA fragment can be selected from the 5′ portion of the HIV genome, for example HIV at nt 455-1514. Although there are many permutations of HIV genomic sequences, an exemplary sequence in this regard is identified as GENBANK nucleotide identification number (NiD) g326382. The sequence will preferably be greater than about 1,000 nucleotides in length and will be subcloned into a transcription vector. A corresponding RNA molecule is then produced by standard in vivo transcription procedures. This is added to the reaction mixture described above, at the beginning of the incubation period. Although the final concentration of RNA molecule present in the mixture will vary, the volume in which such molecule is added to the reaction mixture should be less than about 10% of the total volume.

Preparation of HIV capsids in the cell-free capsid assembly system has revealed the existence of novel previously unrecognized assembly intermediates, and provides means for identification of additional assembly intermediates. As discussed in more detail below, such intermediates are useful as (i) antigens for production of antibodies and/or vaccines, (ii) along with such antibodies, as standards in diagnostic tests, (iii) as vehicles for identification of key host cellular proteins involved in capsid assembly and (iv) drug targets. Exemplified herein are a capsid assembly pathway and intermediates thereof that have been identified for HIV, and similar pathways by analogy are used by other retroviral capsid assembly mechanisms, and that the intermediates described herein have analogous counterparts in such retroviral systems. These counterparts can be identified using the general manipulations described below with respect to HIV.

Capsid assembly intermediates can be formed in a number of ways, including (i) translation of HIV capsid assembly mutant coding sequences in cells or in cell-free preparations, and (ii) by blocking the production of HIV capsids in a cell-free assembly system, such as by adding specific assembly blockers (e.g. apyrase to block ATP, Example 6) or by subtraction of a key component, such as MCoA, from the reaction, resulting in the production of one or more assembly intermediates in large quantity.

At least one host cell-derived assembly protein is involved in capsid formation. The presence of such a protein in a cell extract is detected by any of a number of means, including immunoprecipitation of the complex, as described in Example 4 and Example 6. Alternatively, the protein, such as HP68 can be added exogenously to the system. In studies carried out in support of the present invention, an exemplary host cell assembly protein was found in certain of the capsid complexes described below. This exemplary host cell protein is identified as HP68 and is characterized by (i) immunoreactivity with TCP-1 monoclonal antibody 23c (Inst. for Cancer Research, London, UK; Stressgen, Vancouver, B.C., Canada), and (ii) containing the peptide sequence SEQ ID NO: 2 (PRPYLDVKQRLKAARVIRSLLRSN). Example 6 describes sequencing of the entire open reading frame of the WGHP68, SEQ ID NO: 5, which SEQ ID NO: 2 is a fragment. The protein is further characterized by a molecular weight of 68,000 kilodaltons (as assessed by SDS-PAGE). This protein is distinct from the “detergent insensitive fraction” described in the previous section, as evidenced by the ability of a high speed supernatant of wheat germ extract to block immunoprecipitation of complexes by monoclonal antibody 23c. It is the discovery of the present invention that conformers of HP68 and homologues thereof are cellular agents involved in capsid assembly, and that specific blockade of its reactivity conferred by the conformer may provide new therapeutic regimens for blocking HIV production.

The HP68 conformer can be obtained from any of a variety of sources, including wheat germ and primate homologues, particularly human. Human homologues can be identified using degenerate primers to the HP68 sequence, or other chaperone proteins identified in a cell free system that bind to HIV capsid intermediate complexes, and then cloned into an expression vector. Translation products from these expression vectors are tested in a cell free system to determine their ability to bind HIV capsid assembly proteins by immunopurification.

Host proteins, exemplified by HP68, can be identified that are involved in viral replication which, when present as an alternative conformers, have different activities or functions. For cytosolic proteins, such as HP68, this is accomplished by (i) first producing knockout mice for cytosolic proteins of interest; (ii) generating monoclonal antibodies to probe for conformational specificity, (iii) epitope mapping the conformational; and (iv) in parallel with 1 and 2 characterizatizing the cytosolic proteins in the different complexes and under the conditions that generate one conformer versus another.

Any protein for which evidence suggests conformational heterogeneity can be assessed as a candidate for having a conformer. For example, HP68 is a protein the normal function of which is unknown but which contains an ATP binding site. Decreases in Rnase L inhibitor have also been implicated in chonic fatigue syntrome (reference). However, it has been implicated in two distinct functional assays in viral infection: as a molecular chaperone for viral capsid assembly in HIV infected cells (see Example 5) and as an RNAse L inhibitor (Bisbal et al., JBC (1995) 270(22):13308-17). These activities are mutually exclusive, i.e., the conformer that acts as a chaperone in HIV capsid assembly and binds to Gag does not bind to Rnase L and vice versa. These distinctive functional assays suggest that each conformer occurs in vivo under different circumstances and makes possible the direct determination of conditions that favor one versus the other pathway of biogenesis for nascent HP68 or other candidate conformers. The existence of such conformers makes possible drug targets for inhibiting viral replication that will inhibit a single function of the host protein as opposed to all functions of the host protein if only one conformer is targeted.

Constructs containing cloned cDNA of a suspected conformer can be engineered and expressed in a cell-free system or in transfected mammalian cells (see Example 5 and Hegde et al. Nature (1999) 402:822-826). Radiolabelled amino acid incorporation into specific proteins of interest are assessed by solution immunoprecipitation under native versus denatured conditions and analysis by SDS-PAGE and autoradiography (AR). Nascent chains are analyzed in various ways (e.g. truncation and crosslinking (Hegde et al. Cell (1997) 90:31-41)) to correlate aspects of biogenesis to conformational heterogeneity of the completed polypeptides. The systems, cell free or transfected cells, can be manipulated in various ways (Rutkowski et al. PNAS (2001) 98:7823-7828; Hegde et al. Molecular Cell (1998) 2:85-9) (e.g. viral replication, temperature, energy) and the correlation of effect on biogenesis and effect on final protein conformation can be determined. For cytosolic proteins, analysis will focus on the mechanisms of formation of two (or more) distinct complexes which are readily detected (Sen et al. JBC (1992) 267(8):5017-20; Gorlich et al. Nature (1992) 357:47-52). In addition, the biosynthetic heterogeneity of cytosolic proteins can be characterized and parameters identified that alter the distribution of conformers (see Example 5; and Rutkowski et al., PNAS (2001) 98:7823-7828).

Monoclonal antibodies can be produced to corroborate the functional assay results and show, based on epitope mapping, that (i) antibodies to the same epitopes do not bind proteins that contain essentially the same amino acid sequences; and (ii) alternative folding of proteins masks or uncovers epitopes and renders them immunologically, and thus structurally, distinct. Sequencing of the cloned suspected conformer is conducted to demonstrate that the proteins have essentially the same amino acid sequence. Thus, monoclonal antibodies to a mapped epitope can be used to show that amino acid chains can fold differently under different conditions (e.g. viral replication, temperature, energy), producing conformers with different structural, and, by implication, functional ability. Monoclonal antibodies can be used in unpurified lysates from either transfected cells or a programmed cell-free system.

Monoclonal antibodies can be prepared by any number of means which are known to those skilled in the art and previously described (see, for example, Kohler et al., Nature, 256: 495-497 (1975) and Eur. J. immunol. 6:511-519 (1976); Milstein et al., Nature 266: 550-552 (1977), Koprowski et al., U.S. Pat. No. 4,172,124; Harlow, E. and D. Lane, 1988, Antibodies: A Laboratory Manual, (Cold Spring Harbor Laboratory Press, Cold Spring Harbor, N.Y. (1989); Current Protocols In Moleclular Biology, Vol. 2 (Supplement 27, Summer '94), Ausubel, F. M. et al., Eds., (John Wiley & Sons: New York, N.Y.), Chapter 11, (1991)). Generally, a hybridoma is produced by fusing a suitable immortal cell line (e.g., a myeloma cell line) with antibody-producing cells (for example, lymphocytes derived from the spleen or lymph nodes of an animal immunized with an antigen of interest). The cells resulting from a fusion of immune cells and lymphoma cells, generally referred to as hybridomas, can be isolated using selective culture conditions, and then cloned by limiting dilution. Cells which produce antibodies with the desired binding properties are selected by a suitable assay, such as a serological assay, including enzyme-linked immunosorbent assay (ELISA).

Functional binding fragments of monoclonal antibodies also can be produced by, for example, enzymatic cleavage or by recombinant techniques. Enzymatic cleavage methods include papain or pepsin cleavage to generate Fab or F(ab′)₂ fragments, respectively. Antibodies also can be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH₁ domain and hinge region of the heavy chain.. Functional fragments of the monoclonal antibodies retain at least one binding function and/or modulation function of the full-length antibody from which they are derived. Preferred functional fragments retain an antigen-binding function of a corresponding full-length antibody (e.g., retain the ability to bind an epitope of a conformer). In another embodiment, functional fragments retain the ability to inhibit one or more functions characteristic of a protein or peptide conformer, such as a binding activity, a signaling activity, and/or stimulation of a cellular response. For example, in one embodiment, a functional fragment can inhibit the HIV capsid assembly.

One method of developing conformer specific antibodies is to immunize knock-out mice that lack a functional gene for the protein of interest with a putative conformer of the protein of interest. Knockout mice can be produced using standard techniques known to those skilled in the art (Capecchi, Science (1989) 244:1288; Koller et al. Annu Rev Immunol (1992) 10:705-30; Deng et al. Arch Neurol (2000) 57:1695-1702), for which the gene corresponding to the protein against which monoclonal antibodies will be raised will be knocked out, e.g. HP68. A targeting vector will be constructed which in addition to containing a fragment of the gene to be knocked out will contain an antibiotic resistance gene, preferably neomycin, to select for homologous recombination and a viral thymidine kinase (TK) gene, alternatively the gene encoding diphtheria toxin (DTA) can be used to select against random insertion. The vector is designed so that if homologous recombination occurs the neomycin resistance gene will be integrated into the genome, but TK or DTA gene will always be lost. Murine embryonic stem (ES) cells will be transfected with the linearized targeting vector and through homologous recombination will recombine at the locus of the targeted gene to be knocked out. Murine ES cells will be grown in the presence of neomycin and ganciclovir (for TK), a drug that is metabolized by TK to produce a lethal product. Thus cells that have undergone homologous recombination are resistant to both neomycin and ganciclovir. Vectors contain DTA will kill any cell that codes for the gene, no additional drug is required in the cell culture medium. Southern blotting hybridization and PCR will be used to verify the homologous recombination event, techniques well known to those skilled in the art.

To generate a mouse carrying a disrupted targeted gene, positive ES cells can be propagated in culture to differentiate and a blastocyte to be implanted into a pseudopregnant female, alternatively the ES cells can be injected back into the blastocoelic cavity of a preimplantation mouse embryo and the balstocyte and then surgically implanted. In addition, transfected ES cells and recipient blastocytes will be from mice with different coat colors, so that chimeric offspring can be easily identified. Through breeding techniques homozygous knockout mice will be generated. Tissue from these mice will be tested to verify the homozygous knockout for the targeted gene, again using PCR and Southern blotting hybridization.

In an alternate method, gene targeting using antisense technology can be used (Bergot et al., JBC (2000) 275:17605-17610). The homozygous knockout mice are immunized with purified host protein peptides, both native and denatured recombinant protein. Following subsequent boosts, at 3 and 6 weeks, with the immunogen, the mice are sacrificed and spleens taken and fusion to myeloma cells carried out (Korth et al. Methods in Enzymol. (1999) 309:106). Antibodies from individual hybridomas are screened for conformational specificity, i.e., binding with substantial specificity to a single conformer. The screening process is carried out with radiolabeled protein products produced in the cell-free translation system or radiolabeled media or cell extracts chosen to enrich one versus another conformer. These products are immunoprecipitated using hybridoma supernatant and run on a SDS-PAGE gel. Preferably cell-free extracts are used due to the possibility that the use of transfected cells would result in protein-protein interactions which would block antibodies from binding a specific epitope, thus masking a potential conformer. The use of a solution immunoprecipitation screen with radiolabled translation products, the conformation has been skewed (e.g. by viral infection), is the key that distinguishes this screen from a conventional approach to monoclonal antibody production. The use of 96 well plates for screening streamlines the process, allowing a single technician to screen up to 1000 individual hybridomas in a single day.

For understanding and treating a disease in which host protein or peptide conformers are involved, it is useful to identify one or more antibodies that are substantially specific for a host conformer. This method involves contacting a number of conformers with a number antibodies, or binding fragments derived from specific antibodies. The specificity of binding of the antibodies or fragments to individual conformers is then evaluated. Those antibodies or fragments that are substantially specific for each of the various conformers may thus be identified.

Sequencing of the protein conformers to which monoclonal antibodies have been raised against will show that the conformer proteins contain essentially the same amino acid sequence. Therefore, it is not necessary to develop an epitope map based on linear peptides but instead the protein should be mapped for conformational, or discontinuous, epitopes. The different specificity of the monoclonal antibodies is derived from the different folding of the same amino acid sequence. Thus, conformational epitope mapping is necessary to prove that the monoclonal antibodies are binding to restricted epitopes mapping can also be used to identify the binding sites between capsid proteins in the intermediate examples and host chaperone proteins where in binding sites on the proteins are all potential drug targets. Thus, the identification of these epitopes also has utility in drug targeting.

Discontinuous epitopes can be identified by utilizing limited proteolysis of the antibody bound to a conformer of the protein of interest and then analyzed using mass spectrometry. Monoclonal antibodies (MAb) are bound to a solid support and lysates containing the conformer protein are incubated with the immobilized Mab. Following removal of unbound protein, selected diluted proteases are added to the immobilized Mab-conformer complexes and unbound cleavage products are removed. The bound conformer protein are eluted, under appropriate conditions, and analyzed by LC-MS. Sequencing of the conformer protein and molecular modeling are necessary to fully identify the conformational epitope.

Alternatively, binding between capsid proteins and host proteins in capsid intermediates can be analysed and the binding sites identified using technology developed by Biacore AB (www.biacore.com).

The cell-free system can be used to identify possible compounds that inhibit host proteins necessary for the production of viral particles. Compounds of interest are screened for their ability to inhibit viral replication by blocking host proteins necessary for viral replication. Upon identification of compounds of interest, the compounds are tested in human cells under similar conditions.

Population profiles of conformers associated with disease severity or other characteristics can be developed. The profiles can be developed by contacting a fluid of an individual with HIV, or inflicted cells from the individual, with one or more monoclonal antibodies specific for a unique conformer of a host protein involved in the disease. The fluid may be any body fluid including blood, serum, plasma, lymphatic fluid, urine, sputum, cerebrospinal fluid, or a purulent specimen. A binding fragment derived from a monoclonal antibody specific for a unique conformer may also be used. The monoclonal antibody or binding fragment is labeled with a detectable label, for example, a radiolabel or an enzyme label. Examples of enzyme labels that may be linked to an antibody include horseradish peroxidase, alkaline phosphatase, and urease, and methods for linking enzymes with antibodies are well known in the art. The label may be detected using methods well known to those skilled in the art, such as radiography, or serological methods including ELISA or blotting methods. The presence of the label is indicative of the presence of at least one protein or peptide conformer in the individual, and may be used to identify those conformer profiles that may play a role in the disease process. Detection of the label in a body fluid indicates the presence of at least one protein or peptide conformer in the individual. A plurality of monoclonal antibodies or their binding fragments may be similarly used to detect a plurality of conformers associated with a disease state in an individual.

By detecting and characterizing conformers associated with a disease in a number of individuals in a population, a profile of the various conformers associated with the disease begins to emerge. Establishing a conformer profile in such a population is conducted by detecting and characterizing conformers associated with any given disease in individuals, compiling the data within the population, and then establishing the relationship between conformer profiles of the individual members of the population and specific characteristics of the disease in the individuals. These specific characteristics will depend on the disease and the nature of the protein or peptide conformer. For example, various viral or host protein or peptide conformers may be associated with greater or lesser disease severity. As another example, host protein or peptide conformers may be associated with greater or lesser disease resistance. The response of the individuals within the population to various disease treatments is an important factor in profiling the relationship between the conformer profile of an individual and their responsiveness. Individuals that respond poorly to treatment, for example, may have conformational forms of a protein or peptide involved in the disease process that make poorer targets for the treatment than the conformational forms of the protein or peptide in individuals that respond well to treatment. Generally, population studies are required to establish these relationships between conformers and response with a reasonable degree of significance.

Once a relationship between a conformer and treatment efficacy is established in a population, the selection of a treatment for any given patient can be improved by determining the conformer profile within that patient using, for example, the antibody- or antibody fragment-based methods described above. Those methods that have been established as successful for individuals with substantially similar conformer profiles to that of the patient will be most likely to prove efficacious.

The methods and compositions described herein have a number of useful purposes. For example, the cell-free translation/assembly system for HIV can be used to produce large quantities of the wild-type capsids, capsid intermediates or mutant capsids, as demonstrated in the studies described herein. Such capsids and intermediates can be used, for example to produce vaccines. They also find utility as reagents in screening assays that assess the status of HIV capsid formation or in assays used for screening for drugs that interfere with HIV capsid formation, such as the assay described below.

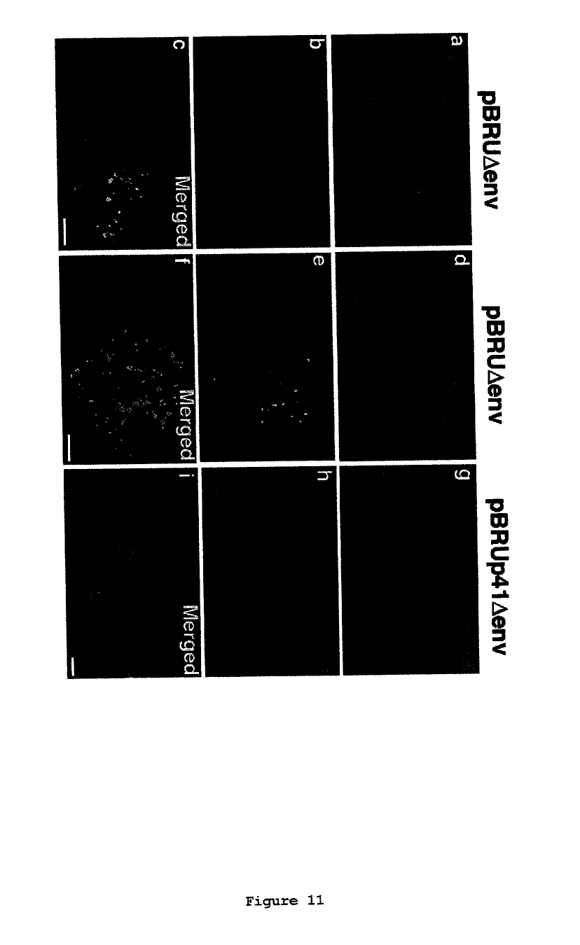

The screening assay of the invention has utility in screening for new drugs for use in the attenuation of HIV infection. The assay can be set up according to any of a number of assay formats. In one such assay, monoclonal or polyclonal antibodies are used directly to screen for compounds that block or impair HIV capsid formation. Preferrably such compounds do not activate host stress responses. As exemplified by immunoflurescent staining in FIG. 11, high throughput screening of compounds for lead candidates that would reverse the distinctive immunofluorescent pattern of Gag and HP68, as seen in FIG. 11, could be conducted. These lead compounds could then be further tested for specificity. In another such assay, cell-free translation and assembly is carried out (in the presence or absence of a candidate drug) in a liquid phase, along the lines of the assay described in Example 1. The reaction product is then added to a solid phase immunocapture site coated with antibodies directed against and specific for one or more of the HIV capsid intermediates or the complete HIV capsid described above. In this way, the precise point of assembly interference of the drug can be determined. Such information should be valuable to clinicians, and drug development companies, particularly in the context of combination therapeutics against HIV infection.

A compound that is found to block HIV capsid formation by binding to the active site would be tested in mammalian cells infected with HIV. Compounds are also screened for toxicity including host stress responses such as activation of heat shock proteins (HSP) 70, 80, 90, 94 and caspases (Flores et al., J. Nueroscience (2000) 20:7622-30). Methods for evaluating activation of these proteins are well known to those skilled in the art. Compounds can first be identified based on searches of databases for compounds likely to bind the active site then tested in a cell-free system for capsid formation. Host cell proteins, exemplified by the HIV specific HP68 conformer, also form a part of the present invention, and have distinctive utilities. This protein from wheat germ extract is identified as being involved in capsid assembly, as evidenced by its association with capsid intermediates, especially intermediates B, C, and D, and is characterized as having a peptide region having the sequence presented as SEQ ID NO: 2 and SEQ ID NO: 5, specific immunoreactivity with monoclonal antibody 23 c, and an apparent molecular weight of about 68 kilodaltons. The protein is characterized by at least 60% amino acid sequence identity to human HP68, herein termed WGHP68. It is appreciated, however, that such a protein can be derived from any of a number of host cell sources, including, but not limited to human cells. The present invention teaches how to identify conformers of host proteins involved in viral replication. Host cell proteins involved in capsid formation or specific antibodies directed to such proteins, can be used to monitor capsid formation. In addition, association of the host protein with specific intermediates can be assayed directly, and such an assay can form a screening assay for drugs that interfere with capsid assembly by interfering with the association of HP68 and HIV Gag and Vif proteins. This can be accomplished by compounds that bind to the active site on either the capsid proteins or host chaperone proteins. Where-in the active site is the binding site on each protein for each other, eg. HP68-Gag.

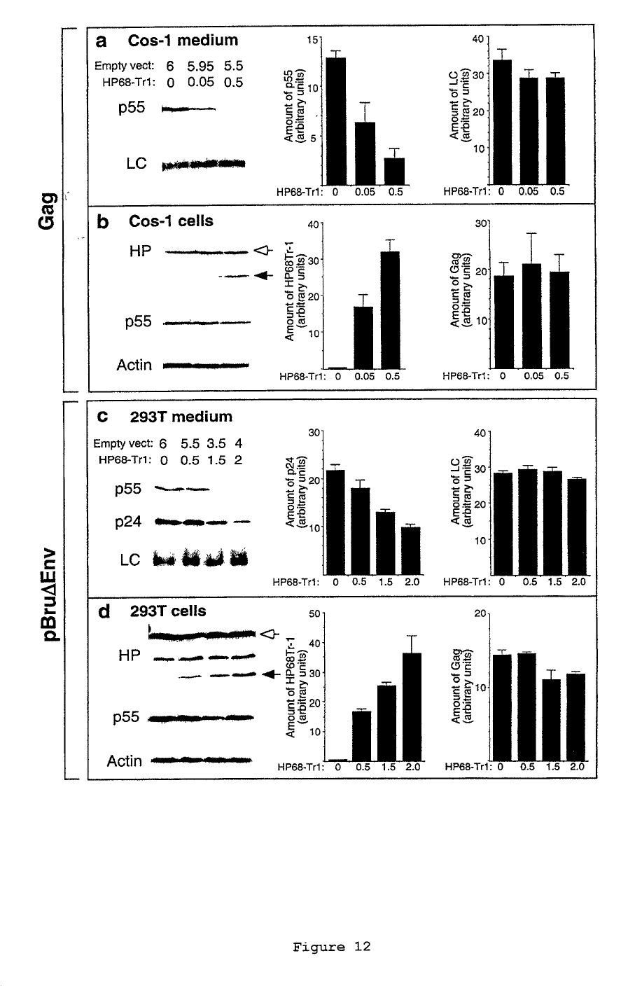

The invention also can be used to identify other host and viral proteins that are involved in regulation of capsid formation. As exemplified in FIG. 12, transfection of an HIV infected mammalian cell with a dominant negative mutant of Gag blocks HIV release. Stably transfected cells can be utilized to screen for other host or viral proteins required for capsid formation by further transfecting these cells with pooled genomic or cDNA clones and screening for clones that are able to restore HIV capsid formation. Thus, clones are selected for their ability to block the HP68 dominant negative mutants from inhibiting viral release from cells.

The invention also can be used as a means of identifying compounds that inhibit HIV capsid formation, by adding to a cell a compound that has been selected for its ability to inhibit capsid formation or formation of capsid intermediate(s) in the cell-free translation system described herein. As a related feature, the invention also extends to provide a method of selecting compounds effective to alter HIV capsid formation in cells. According to this feature of the invention, the test compound is added to cells that are forming HIV retroviral capsids. The quantity and nature of capsid intermediates formed is measured and compared to capsids formed in control cells. The compound is selected if the quantity or nature of intermediates measured in the presence of the compound is significantly different than those formed in the absence of the compound. Association of host assembly protein HP68 with capsid intermediates can be used as a measurement in such a selection method, as well.

The cell free system can be used with plasmids that code for the entire HIV genome, except for envelope protein. Thus, the invention includes a method of encapsidating genomic HIV RNA or fragments thereof Genomic HIV RNA, RNA fragment or a plasmid encoding HIV RNA is added to such a system, and is encapsidated during the reaction process.

The following examples illustrate, but in no way are intended to limit the present invention.

### EXAMPLES

### Materials

1. Chemicals

Chemical sources are as follows, unless otherwise indicated below: Nonidet P40 (NP40) was obtained from Sigma Chemical Co. (St. Louis, Mo.). “NIKKOL” was obtained from Nikko Chemicals Ltd. (Tokyo, Japan). Wheat Germ was obtained from General Mills (Vallejo, Calif.). Myristoyl Coenzyme A (MCoA) was obtained from Sigma Chemical Co. (St. Louis, Mo.).

2. Plasmid Constructions

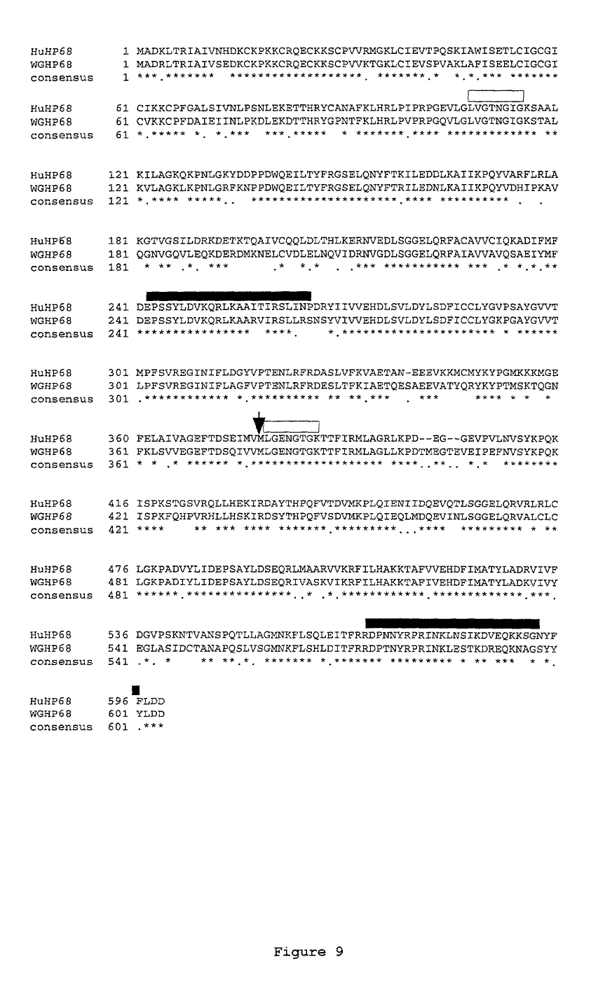

All plasmid constructions for cell-free transcription were made using polymerase chain reactions (PCR) and other standard nucleic acid techniques (Sambrook, J., et al., in Molecular Cloning. A Laboratory Manual). Plasmid vectors were derived from SP64 (Promega) into which the 5′ untranslated region of Xenopus globin had been inserted at the Hind III site (Melton, D. A., et al., Nucleic Acids Res. 12:7035-7056 (1984)). The gag open reading frame (ORF) from HIV genomic DNA (a kind gift of Jay Levy; University of California, San Francisco) was introduced downstream from the SP6 promoter and the globin untranslated region. The GΔA mutation was made by changing glycine at position 2 of Gag to alanine using PCR (Gottlinger, H. G., et al., Proc. Natl. Acad. Sci. 86:5781-5785 (1989)). The Pr46 mutant was made by introducing a stop codon after gly 435 (removes p6); Pr41 has a stop codon after arg 361 (in the C terminal region of p24). These truncation mutants are comparable to those described by Jowett, J. B. M., et al., J. Gen. Virol. 73:3079-3086 (1992), incorporated herein by reference. To make the D2 mutant amino acids from gly 250 to val 260 were deleted (as in Hockley, D. J. et al., J. Gen. Virol. 75:2985-2997 (1994); Zhao, Y., et al., Virology 199:403-408 (1994)). All changes engineered by PCR were verified by DNA sequencing. The plasmid, pBRUΔenv, which encodes for the entire HIV-1 genome except a deletion in envelope, was made and used as previously described (Kimpton et al. J. Virology (1992) 66:2232-9). The plasmid, WGHP68-Trl, encodes a 379 amino acid truncated form of HP68 with a stop codon before the second nucleotide-binding domain (Arrow, FIG. 9). This plasmid encodes the N-terminal two-thirds of WGHP68 and produces the expected 43 kD protein when transfected into cells (FIG. 12)

3. 35-S Energy Mix

35-S Energy Mix (5× stock) contains 5 mM ATP (Boehringer Mannheim), 5 mM GTP (Boehringer Mannheim), 60 mM Creatine Phosphate (Boehringer Mannheim), 19 amino acid mix minus methionine (each amino acid except methionine; each is at 0.2 mM), 35-S methionine 1 mCurie (ICN) in a volume of 200 microliters at a pH of 7.6 with 2 M Tris base.

4. Compensating Buffer

The Compensating Buffer (10X) contains 40 mM HEPES-KOH, at a pH of 7.6 (U.S. Biochemicals), 1.2 M KAcetate (Sigma Chemical Co.), and 2 mM EDTA (Mallinckrodt Chemicals, Paris, Ky.).

### Example 1

### Cell Free Protein Synthesis

1. Transcription

The plasmid containing the Gag coding region was linearized at the EcoRi site (as described in the NEB catalogue). The linearized plasmid was purified by phenol-chloroform extraction (as described in Sambrook, J., et al., in Molecular Cloning. A Laboratory Manual) and this plasmid was adjusted to a DNA concentration of 2.0 mg/ml. Transcription was carried out using a reaction that contained: 40 mM Tris Ac (7.5), 6 mM Mg Ac, 2 mM Spermidine, 0.5 mM ATP, 0.5 mM CTP, 0.5 mM UTP, 0.1 mM GTP, 0.5 mM diguanosine triphosphate (cap), 10 mM Dithiothreitol, 0.2 mg/ml transfer RNA (Sigma Chemical Co.), 0.8 units/microliter RNAse inhibitor (Promega), 0.4 units per microliter of SP6 Polymerase (NEB). Mutant DNAs were prepared as described by Gottlinger, H. G., et al., Proc. Natl. Acad. Sci. 86:5781-5785 (1989); Jowett, J. B. M., et al., J. Gen. Virol. 73:3079-3086 (1992); Hockley, D. J. et al., J. Gen. Virol 75:2985-2997 (1994); or Zhao, Y., et al., Virology 199:403-408 (1994); these publications are incorporated herein by reference.

2. Translation

Translation of the transcription products was carried out in wheat germ extract containing ³⁵S methionine (ICN Pharmaceuticals, Costa Mesa, Calif.). The wheat germ extract was prepared as described by Erickson and Blobel (1983) as modified below. Reactions were performed as previously described (Lingappa, J. R., et al., J. Cell. Biol. (1984) 125:99-111), except for modifications noted below.

A 25 microliter wheat germ transcription/translation reaction mixture contained: 5 microliters Gag transcript (prepared as described in transcription methods), 5 microliters wheat germ extract (prepared as described in wheat germ preparation; preferably using the high speed supernatant detailed in Example 4), 5 microliters 35-S Energy Mix 5× stock (Sigma Chemical Co., St. Louis, Mo.), 2.5 microliters Compensating Buffer (Sigma Chemical Co.), 1.0 microliter 40 mM MgAcetate (Sigma Chemical Co.), 2.0 microliters 125 5M Myristoyl CoA (made up in 20 mM Tris Acetate, pH 7.6; Sigma Chemical Co.), 3.75 microliters 20 mM Tris Acetate buffer, p11 7.6 (U.S. Biochemicals; Cleveland, Ohio), 0.25 microliter Creatine Kinase (4 mg/ml stock in 50% glycerol, 10 mM Tris Acetate; Boehringer Mannheim, Indianapolis, Ind.), 0.25 microliter bovine tRNA (10 mg/ml stock; Sigma Chemical Co.), and 0.25 microliter RNAse Inhibitor (20 units/50; Promega).

3. Preparation of Wheat Germ Extract

Wheat germ was obtained from General Mills. Wheat germ extract was prepared as described by Erickson and Blobel (1983) with indicated modifications. Three grams of wheat germ were placed in a mortar and ground in 10 ml homogenization buffer (100 mM K-acetate, 1 mM Mg-acetate, 2 mM CaCl₂, 40 mM HEPES buffer, pH 7.5 (Sigma Chemicals, St. Louis, Mo.), 4 mM dithiothreitol) to a thick paste. The homogenate was scraped into a chilled centrifuge tube and centrifuged at 4° C. for 10 min at 23,000×g. The resulting supernatant was centrifuged again under these conditions to provide an S23 wheat germ extract.

Improved assembly was obtained when the S23 wheat germ extract was further subjected to ultracentrifugation at 50,000 rpm in the TLA 100 rotor (100,000×g) (Beckman Instruments, Palo Alto, Calif.) for 15 min at 4° C. and the supernatant used for in vitro translation. This improvement provided 2-3× the yield obtained in comparable reactions using the S23 wheat germ extract. This supernatant is referred to herein as a “high speed wheat germ extract supernatant”. It is appreciated that extracts of other eukaryotic cells, such as rabbit reticulocytes may be used to form analogous high-speed supernatants, and that such supernatants will be useful in practicing the present invention.

Myristoyl coenzyme A (MCoA; Sigma, St. Louis, Mo.) was added at a concentration of 10 micromolar at the start of translation when indicated. Translation reactions ranged in volume from 20 to 100 microliters and were incubated at 25° C. for 150 min. Some reactions were adjusted to a final concentration of the following agents at tunes indicated in the figures and specification: 0.2 μM emetine (Sigma); 1.0 units apyrase (Sigma) per mL translation; 0.002%, 0.1%, or 1.0% “NIKKOL”. Cell-free translation and assembly reactions were also carried out successfully in rabbit reticulocyte lysate prepared as described previously (Merrick, W. C., Methods Enzymol. 101 :606-615 (1983)) or obtained from commercial suppliers (Promega, Madison, Wis.). In pulse-chase experiments, translation reactions contained ³⁵S cysteine (Amersham Life Sciences, Cleveland, Ohio) for radiolabeling. After 4 min translation reaction time, 3 mM unlabeled cysteine was added, and the reaction was continued at 25° C. for variable chase times as indicated in the experiments described herein.

4. Estimation of Sedimentation Coefficients

Estimates of S-values of Gag-containing complexes seen on 13 ml sucrose gradients were determined by the method of McEwen, C. R., Anal. Biochem. 20:114-149 (1967) using the following formula:

S=ΔAI/ω²t

where S is the sedimentation coefficient of the particle in Svedberg units, ΔI is the time integral for sucrose at the separated zone minus the time integral for sucrose at the meniscus of the gradient, ω is rotor speed in radians/sec. and t is time in sec.

Values for I were determined for particles of a density of 1.3 g/cm3 and for a temperature of 5° C., according to tables published by McEwen, C. R., Anal. Biochem. 20:114-149 (1967). Calculated S values for different fractions in the gradients are labeled as markers above each gradient tracing shown herein. Markers such as BSA (5-S), macroglobulin (20-S), Hepatitis B Virus capsids (100-S), ribosomal subunits (40-S and 60-S), and polysomes (>100-S) were used to calibrate the gradients and to confirm the calculated S values. However, it should be noted that the S value assignments for each Gag-containing complex are approximate estimates and may vary by about ±10%.

### Example 2

### Preparation of HSS, HSP, and HSPd

Where indicated, wheat germ extract prepared as described in Example 1 was centrifuged at either 50,000 rpm for 21 min or 100,000 rpm for 30 min in a TLA 100 rotor (Beckman Instruments, Palo Alto, Calif.). The supernatant (high-speed supernatant, HSS) of the 50,000 rpm spin was used for cell-free translation and assembly reactions. The pellet of the 100,000 rpm spin (high speed pellet, HSP) was resuspended at a 5× concentration in buffer (25 mM Hepes pH 7.4, 4 mM MgAc, 100 mM KAc, 0.25M sucrose). Wheat germ extract adjusted to contain a concentration 0.5% “NIKKOL” was subjected to the same ultracentrifugation in parallel to generate the detergent treated high-speed pellet (HSPd). This pellet was washed twice with 200 μL of the above non-detergent buffer in order to remove traces of detergent, and then resuspended as described above. Following treatment with emetine at 50 mm, 1.8 μL of HSP or HSPd was added to the 18 mL cell-free reactions programmed with HSS. Control reactions were treated with the same volume of buffer at the same time. At the end of the 150 min incubation, reactions were separated into soluble and particulate fractions and analyzed as described above.

### Example 3

### Translation of Gag Pr55 Protein in a Cell Free System

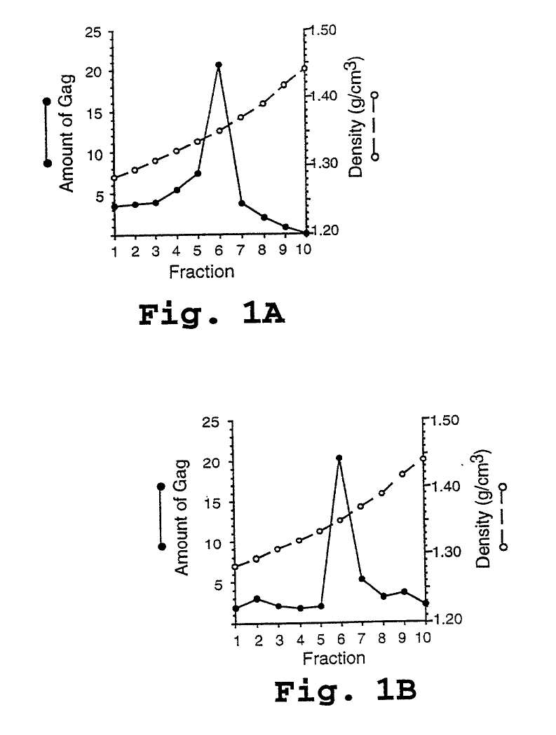

The cell-free translation/assembly system of the invention contains the components described in Part A, above. Example 1 provides details of an exemplary system derived from wheatgerm extract, which is capable of supporting translation and assembly of HIV capsids. Briefly, protein synthesis was initiated in the cell-free translation/assembly system by adding an mRNA that encodes Gag Pr55 protein. Alternatively, when the system includes transcription means, such as SP6 or T7 polymerase, the reaction may be initiated by addition of DNA encoding the protein. Complete synthesis of protein and assembly into capsids is usually achieved within about 150 minutes. FIG. 1 shows that capsids formed in the cell-free system of the invention are substantially the same as those formed in cells. Shown in the Figure is a comparison of migration of the capsids through an isopycnic CsCl gradient, where capsids formed in the cell-free translation/assembly system are shown in FIG. 1A, and capsids formed in transfected Cos cells are shown in FIG. 1B. Cell-free translation and assembly reactions containing 10 μM MCoA and ³⁵S methionine were programmed with HIV Gag transcript and incubated under the conditions detailed in Example 1. At the end of the reaction, samples were diluted into buffer containing 1% NP40 (a non-ionic detergent), and separated into soluble and particulate fractions on sucrose step gradients, according to standard methods known in the art employing sucrose step or linear gradients as appropriate. The particulate fraction was collected and analyzed by velocity sedimentation on a 13 -ml 15-60% linear sucrose gradient (Beckman SW40 Ti rotor, 35,000 rpm, 75-90 min). Fractions from the gradient were collected and subjected to sodium lauryl sulfate polyacrylamide gel electrophoresis (SDS-PAGE) analysis according to standard methods.

A parallel analysis of the particulate fraction was performed by subjecting the particulate fraction to CsCl gradient separation (2 ml isopycnic CsCl, 402.6 mg/ml; 50,000 rpm in a Beckman TLA 100 centrifuge) according to standard methods. Fractions were collected and assessed for Gag translation product (Pr55) (top of gradient is fraction 1, open circles, FIG. 1B). The fractions containing radiolabeled Pr55 were also subjected to SDS PAGE analysis; Gag content of the various fractions was estimated by scanning densitometry of autoradiographs made from the gels. Both conditions produced identical radiolabeled protein bands under these conditions. Material in the particulate fraction (>500-S) was further analyzed by a variety of methods as described below.

Translation of the HIV Gag transcript encoding Pr55 in the cell-free system resulted in the synthesis of approximately 2 ng Pr55 protein per microliter translation reaction. It is appreciated that increased production might be achieved, for example, by employing a continuous flow translation system (Spirin, A. S., et al., Science 242: 1162-1164 (1988)) augmented with the specific factors and components described above.

### Example 4

### Transfections and Production of Authentic Capsids

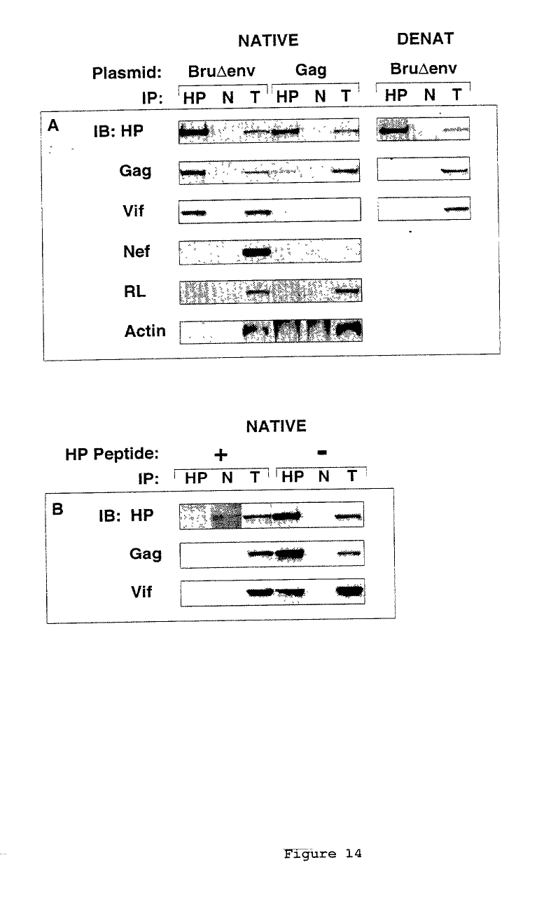

Cos-1 cells (University of California Cell Culture Facility) were transfected by the adenovirus-based method (Forsayeth, J. R. and Garcia, P. D., Biotechniques 17:354-358 (1994)), using plasmids pSVGagRRE-R (a mammalian expression vector that encodes Gag as well as the Rev response element required for expression of Gag in mammalian cells) and pSVRev (a mammalian expression vector that encodes the Rev gene, the product of which is required for expression of Gag in mammalian cells) (Smith, A. J., et al., J. Virol. 67:2266-2275 (1993)). These vectors were provided by D. Rekosh (University of Virginia). Cells were also transfected with pBRUΔenv, FIG. 14. Four days after transfection, immature HIV particles were purified from the culture medium by sedimentation through a 4 ml 20% sucrose cushion in an SW 40 rotor at 29,000 rpm for 120 min (Mergener, K., et al., Virology 186:25-39 (1992)). The pellet was harvested, stored in aliquots at −80° C., and treated with 1% NP40 buffer just before use to remove envelopes. These de-enveloped authentic immature HIV capsids were used as standards and analyzed in parallel with the products of cell-free reactions by a variety of methods, including velocity sedimentation, equilibrium centrifugation, and electron microscopy.

Detergent-treated capsids generated in the cell-free system and detergent-treated (de-enveloped) authentic capsids behaved as a relatively homogenous population of particles of approximately 750-S (compare FIGS 1A and 1B), with a buoyant density of 1.36 g.cm-3. Additionally, cell-free-assembled capsids and the authentic standard were identical in size as judged by gel filtration. Electron microscopic analysis revealed that capsids made in the cell-free system were morphologically similar to authentic capsids released from transfected cells and had the expected diameter of approximately 100 nm (Gelderblom, H. R., AIDS 5:617-638 (1991)). Thus, radiolabeled Pr55 protein synthesized in the cell-free system assembles into particles that closely resemble authentic immature HIV capsids generated in transfected cells, as judged by EM appearance as well as the biochemical criteria of size, sedimentation coefficient, and buoyant density.

A lysate of transfected Cos cells was prepared by solubilizing transfected cells on 60 mm plates in 700 μL 1% NP40 buffer. This detergent lysate was passaged 20 times through a 20-gauge needle, clarified by centrifugation for 10 min at 2000×g, and 150 mL of this supernatant was loaded onto 13 ml sucrose gradients for analysis as described in Example 2. Gag polypeptide present in the fractions was visualized by immunoblotting with a monoclonal antibody to Gag (Dako, Carpenteria, Calif.). Bound antibody was detected using an enhanced chemiluminescence system (Amersham). Band density was determined as described under image analysis below, and relative band densities were confirmed by quantitating films representing different exposure times.

### Example 5

### Immunoprecipitation of Capsid Assembly Intermediates

Immunoprecipitation under native conditions was performed by diluting 2 μL samples of cell-free reactions into 30 μL of 1% NP40 buffer, and adding approximately 1.0 μg of one of monoclonal antibody 23 c (Institute for Cancer Research, London, UK; Stressgen, Vancouver, BC). Samples containing antibodies were incubated for one hour on ice, a 50% slurry of Protein G beads (Pierce, Rockford, Ill.) or Protein A Affigel (BioRad, Richmond, Calif.) was added, and incubations with constant mixing were performed for one hour at 4° C. Beads were washed twice in 1% NP 40 buffer containing 0.1 M Tris, pH 8.0, and then twice in wash buffer (0.1 M NaCl, 0.1 M Tris, pH 8.0, 4 mM MgAc). Proteins were eluted from the beads by boiling in 20 μL SDS sample buffer and were visualized by SDS-PAGE and autoradiography, according to methods well known in the art.

### Example 6

### Requirements of Capsid Assembly

1. Myristoylation of Pr55

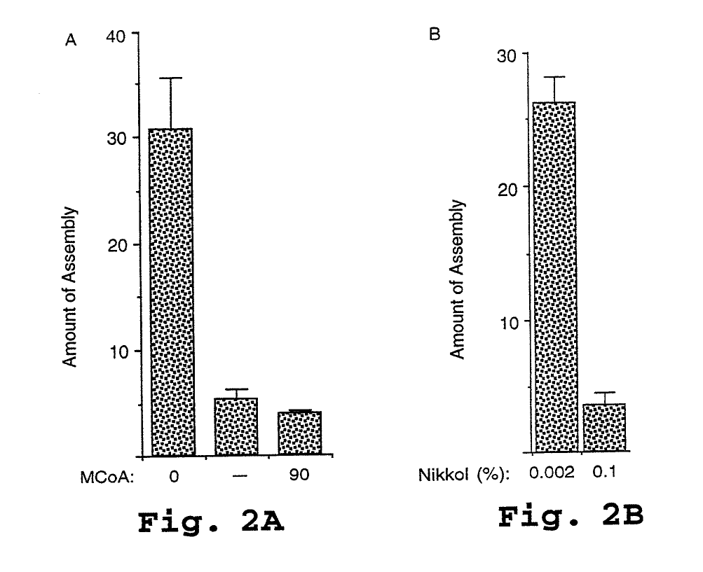

FIGS. 2A and 2B show the results of experiments carried out in support of the present invention in which the cell-free translation/assembly reaction was run in the absence or presence of certain components. FIG. 2A shows the effects of addition of myristoyl coenzyme A (MCoA) to a cell-free translation and assembly reaction programmed with Gag transcript. As shown, the reaction was run in the absence of added MCoA (“−”) or with 10 μM MCoA added either at the start of the reaction (“0”) or at 90 minutes into the reaction when translation is completed (“90”). The detergent-treated products of the cell-free reactions were separated into soluble and particulate fractions by centrifugation on step gradients, and radiolabeled protein in each fraction was visualized by SDS-PAGE and AR as described above. The amount of radiolabeled Pr55 in the particulate fraction (which contains assembled capsids) was determined by densitometry of bands and is expressed as percent of total Gag protein synthesized. The presence of MCoA had no effect on the total amount of Pr55 synthesized; however, it did affect the amount of assembly into capsids, as shown. In the absence of MCoA, or when MCoA was not added until late in the reaction at a post-translational phase (90 min), very little assembly occurred. Values shown are the average of 3 independent experiments, and error bars indicate standard error.

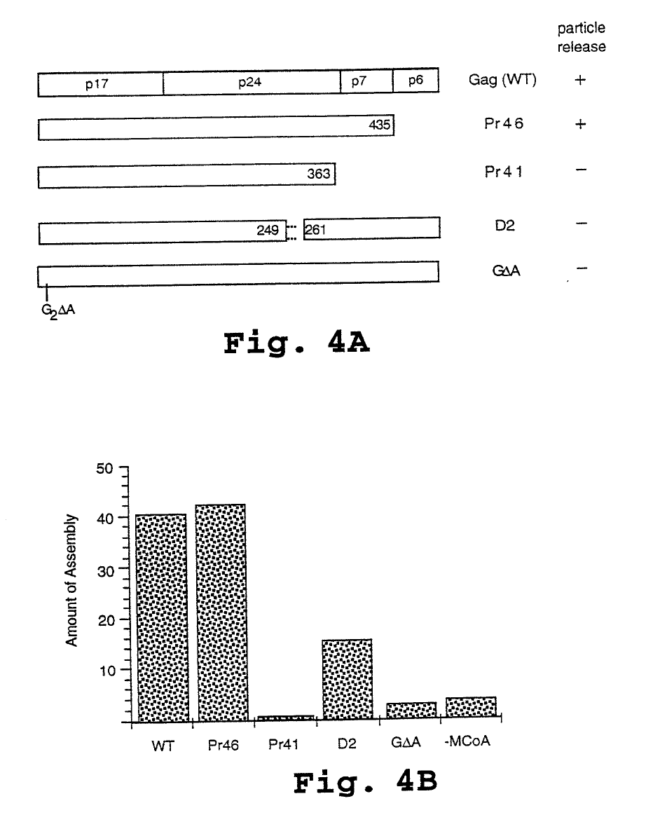

Without ascribing to any particular underlying mechanistic theory, the foregoing results suggest that capsid assembly in the cell-free system requires co-translational myristoylation. This is consistent with an N-terminal modification of the protein which may be required for interaction of the assembly proteins with the inner aspect of a plasma membrane fraction that is required for assembly (Gheysen, D. et al., Cell 59:103-112 (1989); Bryant and Ratner, 1990; Wang, C. -T. and Barklis, E., J. Virol. 67:4264-4273 (1993); Platt, E. J. and Haffar, O. K., Proc. Natl. Acad Sci. 91:4594-4598 (1994); Spearman, P. et al., J. Virol. 68:3232-3242 (1994); Hockley, D. J. et al., J. Gen. Virol. 75:2985-2997 (1994); Bryant and Ratner, 1990; Jacobs E., et al., Gene 79:71-81 (1989). Consistent with these data, in experiments carried out in support of the present invention, a Gag mutant that fails to become myristoylated (GΔA) is also incapable of assembly in the cell-free system (see FIG. 4B).

2. Detergent-Sensitive Component

Studies carried out in support of the present invention have revealed that another critical component of the HIV capsid formation is sensitive to detergent concentrations above the critical micelle concentration (cmc). Membrane fragments are present in the exemplary wheat germ extracts used in experiments described herein, as evidenced by sensitivity of the reaction to addition of detergent at concentrations that solubilize membranes.

Solubilization of membranes can be effected by addition of the detergent “NIKKOL” (octaethyleneglycol mono n-dodecyl ether; Nikko Chemical Co., Tokyo, Japan) at a concentration of 0.1%. At this concentration, “NIKKOL”, a relatively gentle non-ionic detergent, had no effect on Gag polypeptide synthesis. However, as shown in FIG. 2B, “NIKKOL” at this concentration largely abolished capsid assembly. In the experiments shown, cell free translation and assembly reactions containing 10 μM MCoA were programmed with Gag transcript. “NIKKOL” was added at the start the translation reaction to a final concentration of 0.002 or 0.1%, as indicated. At the end of the incubation, the reactions were analyzed for amount of assembly as described above in relation to FIG. 2A. Values shown are the average of 3 independent experiments, and error bars indicate standard error. This effect was not observed when “NIKKOL” was used at a concentration of 0.002%, which is below that required to disrupt lipid bilayers (Walter, P. and Blobel, G., Proc. Natl. Acad. Sci. U.S.A. 77:7112-7116 (1980)).

In further experiments carried out in support of the invention, it was found that “NIKKOL” added after the completion of the 150 min. assembly reaction did not diminish the amount of assembly, even when added to a concentration of 1.0%. Thus, it appears that whereas the integrity of the completed capsid shell is not sensitive to “NIKKOL” (even at high concentrations), assembly of this structure is inhibited by concentrations of “NIKKOL” that are sufficient to solubilize membranes. Further, as described in more detail below, when the Pr55 translation/assembly reaction was treated with emetine and 0.1% “NIKKOL” during a post-translational phase 50 min into the reaction, assembly was dramatically reduced.

The foregoing data are consistent with the idea that membranes are required for newly-synthesized and myristoylated Pr55 chains to be assembled efficiently into capsids in the cell-free system.

3. Incubation Conditions

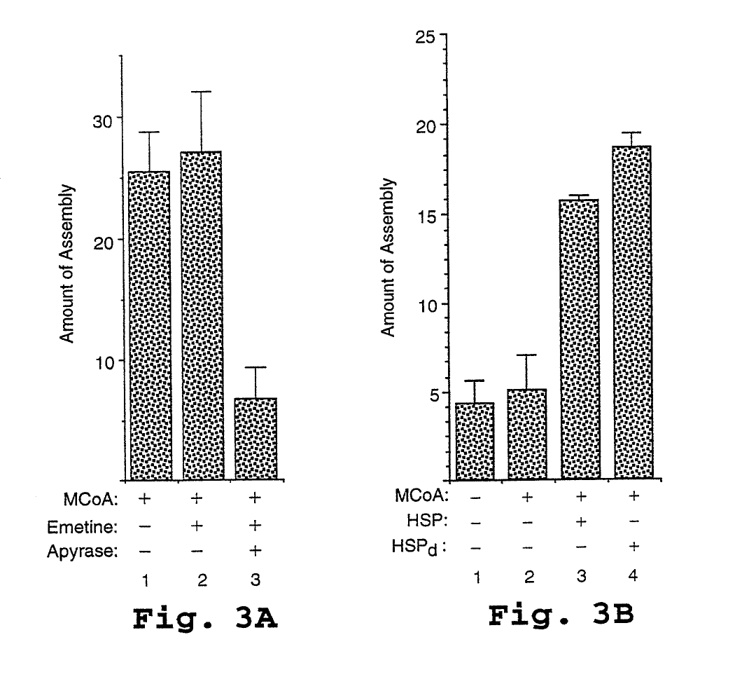

In experiments carried out in support of the present invention, it was found that optimum assembly in the cell-free system requires incubation at 25° C. for at least 150 min, though it is appreciated that these conditions can be varied somewhat while still obtaining translation and assembly. Most Pr55 synthesis occurs during the first hour of this incubation; significant capsid formation does not take place until the final 90 min of the reaction. Thus, an aliquot of the reaction incubated for only 50 min contains approximately 60% of the full-length Pr55 chains that are present in an aliquot incubated for the standard 150 min. However, essentially none of the chains present at the 50 min time point have assembled into caps ids, while at 150 min 25% have completed the assembly process (see FIG. 3A).

Based on these observations, it was possible to separate the translation and assembly phases of the reaction. To confirm this, a reaction mixture was split into two aliquots after 50 min incubation time. To one aliquot emetine was added. (Emetine blocks translation by inhibiting chain elongation.) Both aliquots were incubated to the 150 min time point. While total Pr55 synthesis in the emetine-treated reaction was 60% of the control, the proportion of capsid assembly in this treated reaction was comparable to that of the untreated control (FIG. 3A, bar graph), indicating that assembly takes place even when translation is halted. These data provide basis for dividing the reaction into two phases, where manipulations performed after emetine treatment are observed to have effects on only the post-translational phase of assembly and should not affect Pr55 synthesis, which is already completed.

4. Energy Requirement

According to an important aspect of the invention, assembly of capsids is dependent upon the presence of an energy source in the reaction mixture. An exemplary energy source is the creatine phosphate-creatine phosphokinase system, which regenerates ATP. Equivalent energy sources will be known to those skilled in the art. In experiments carried out in support of the invention, cell-free translation and assembly reactions were programmed with Pr55 in the presence of 10 μM MCoA. Gag translation was allowed to proceed for 50 min, at which point further protein synthesis was inhibited by addition of 0.2 μM emetine. Immediately after emetine treatment, apyrase, an enzyme that hydrolyzes ATP, was added at a concentration of 1 unit/microliter to one of the emetine-treated reactions. At the end of the incubation (150 min), 1 μl of each reaction was analyzed directly by SDS PAGE (autoradiographs are shown below bar graph). The remainder of the products were analyzed for amount of assembly as described above. Shown in the bar graph is the amount of Pr55 assembled as a percent of total Pr55 synthesized in each reaction. Values in the bar graph are the average of 3 independent experiments, and error bars indicate the standard error.

Depletion of free ATP from the assembly reaction by apyrase treatment resulted in a dramatic reduction in capsid assembly (FIG. 3A, bar graph). The effect of ATP depletion was not reversed by addition of the non-hydrolyzable analogue AMP-PNP after apyrase treatment, suggesting that ATP hydrolysis, and not just ATP binding, is required. Addition of apyrase did not change the total amount of Pr55 synthesis, as assessed by measurement of amount of protein by SDS-PAGE analysis, confirming that the effect was on capsid assembly rather than on protein translation. Furthermore, adding apyrase to the reaction after capsid assembly was completed had no effect on the amount of assembly, indicating that the ATP depletion did not affect capsid stability. These data indicate that there is a requirement for an energy source such as ATP in the capsid assembly process, and that this ATP dependence is distinct from the energy requirements of protein synthesis.

5. Detergent-Insensitive Subcellular Component

According to another feature of the invention, it was found that reconstitution of the reaction mixture with a subcellular fraction promotes assembly. As described below this component is distinguished by its relative insensitivity to detergent. Specifically, it is not inactivated by exposure to 0.5% “NIKKOL”.

Wheat germ extract was subjected to ultracentrifugation as described in Example 2 to generate the high-speed supernatant (HSS, depleted of components having sedimentation velocities of 90S or greater), high-speed pellet (HSP), and detergent-treated high speed pellet (HSPd). The HSS was used to program cell-free translation and assembly reactions in the presence or absence of 10 μM MCoA (as indicated in FIG. 4B). Each of these reactions was treated with the protein synthesis inhibitor emetine at 50 min. Following this, the HSP or HSPd was added to aliquots of the reaction as indicated below the bar graph in FIG. 3B. All reactions were incubated for a total of 150 min. A one microliter aliquot was removed and analyzed directly by SDS PAGE (shown below bar graph in FIG. 3B). The remainder of each reaction was analyzed for amount of assembly as described above and plotted as percent of total Pr55 present in each reaction. The values shown in the bar graph are the average of 3 independent experiments, and error bars indicate the standard error.

These experiments showed that the HSS, depleted of components that were 90-S or greater, supported Pr55 translation but not its assembly (FIG. 3B). This indicates that the HSP likely contains assembly-specific host factor(s). This was demonstrated directly by showing that addition of the HSP post-translationally (following emetine treatment) to unassembled Gag chains synthesized in the HSS resulted in a considerable restoration of particle assembly (FIG. 3B). In these experiments, total synthesis of Pr55 was unaltered by addition of the HSP. Together, these data indicate that a subcellular fraction of the eukaryotic cell lysate is required for post-translational events in capsid assembly to take place. That this component is distinct from the plasma membrane component described above is evidenced by the experiments described below indicating that, unlike the plasma membrane component, this component is not sensitive to treatment with a non-ionic detergent.

HSP was examined for the presence of a detergent-sensitive component that is required for capsid formation. HSP was prepared from a cell extract treated with detergent (0.5% “NIKKOL”). The resulting HSP (“HSP_{d}”) was washed with detergent-free buffer, and was added post-translationally to an assembly reaction. As shown in FIG. 3B, HSP from the detergent-treated extract was equally as active in promoting post-translational capsid formation as the control HSP (FIG. 3B, bar graph). Thus, separate detergent-sensitive and detergent-insensitive host factors appear to be involved in the posttranslational phase of HIV capsid assembly. Furthermore, the detergent-insensitive host factor can be depleted by ultracentrifugation and then reconstituted by post-translational addition. According to a further feature of the invention it is appreciated that the detergent-insensitive subcellular component can be further fractionated and characterized.

### Example 7

### HIV Mutant Capsid Formation

Studies of capsid assembly in cultured cells have revealed that certain mutations within the Gag coding region disrupt immature HIV capsid assembly. Four previously-described mutations in Gag are diagrammed in FIG. 4A: (i) the Pr46 mutant, in which the C terminal p6 domain of Gag is deleted (Jowett, J. B. M., et al., J. Gen. Virol. 73:3079-3086 (1992); Spearman, P. et al., J. Virol. 68:3232-3242 (1994); Royer, M., et al., Virology 184:417-422 (1991); Hockley, D. J. et al., J. Gen. Virol. 75:2985-2997 (1994); (ii) the Pr41mutant, in which the deleted domains include p6, the entire nucleocapsid region (p7), and the distal end of p24 containing the p24-p7 protease cleavage site (Gheysen, D. et al., Cell 59:103-112 (1989); Jowett, J. B. M., et al., J. Gen. Virol. 73:3079-3086 (1992); Hockley, D. J. et al., J. Gen. Virol. 75:2985-2997 (1994); (iii) the D2 mutation, in which 10 amino acids of the p24 domain of Gag (upstream of the p24-p7 protease cleavage site) are Zhao, Y., et al., Virology 199:403-408 (1994); Hockley, D. J. et al., J. Gen. Virol. 75:2985-2997 (1994); and (iv) the GAA mutation, an N-terminal single amino-acid substitution that abolishes myristoylation of Gag (Gottlinger, H. G., et al., Proc. Natl. Acad. Sci. 86:5781-5785 (1989); Bryant and Ratner, 1990). Upon expression in cells, only the Pr46 mutant was capable of producing viral particles indistinguishable from those produced by expression of wild-type Gag (Jowett, J. B. M., et al., J. Gen. Virol. 73:3079-3086 (1992); Spearman, P. et al., J. Virol. 68:3232-3242 (1994); Royer, M., et al., Virology 184:417-422 (1991); Hockley, D. J. et al., J. Gen. Virol. 75:2985-2997 (1994). Expressions of each of the other three mutations fails to result in efficient viral particle production and release (Gheysen, D. et al., Cell 59:103-112 (1989); Jowett, J. B. M., et al., J. Gen. Virol. 73:3079-3086 (1992); Hockley, D. J. et al., J. Gen. Virol. 75:2985-2997 (1994); Zhao, Y., et al., Virology 199:403-408 (1994); Gottlinger, H. G., et al., Proc. Natl. Acad. Sci. 86:5781-5785 (1989); Bryant and Ratner, 1990).

FIG. 4A shows schematically the Gag polyprotein precursor that consists of four domains, referred to as p17, p24, p7, and p6, and the mutants discussed above. The Pr46 and Pr41 mutants were constructed by introducing a stop codon truncation at amino acid 435 or at amino acid 363, respectively. In the D2 mutation, amino acids 249 to 261 are deleted. In the GΔA mutation, the glycine at amino acid 2 is substituted with an alanine, thereby blocking myristoylation. The known phenotypes with respect to particle release from cells expressing each of these mutants is indicated to the right (for references, see text).

FIG. 4B shows capsid assembly in cell-free reactions programmed with Gag mutants. Cell-free translation and assembly reactions were programmed with transcript coding for each of the Gag mutants described above, as well as transcript coding for wild-type Gag in the presence or absence of MCoA (labeled WT and -MCoA, respectively). At the end of the reaction period, each sample was detergent treated, fractionated on velocity sedimentation on 13 ml sucrose gradients, and analyzed by SDS-PAGE and autoradiography. The amount of radiolabeled translation product in the position of completed 750S capsids was quantitated by densitometry and expressed for each reaction as % of total synthesis. The total amount of translation was approximately equal in all reactions.

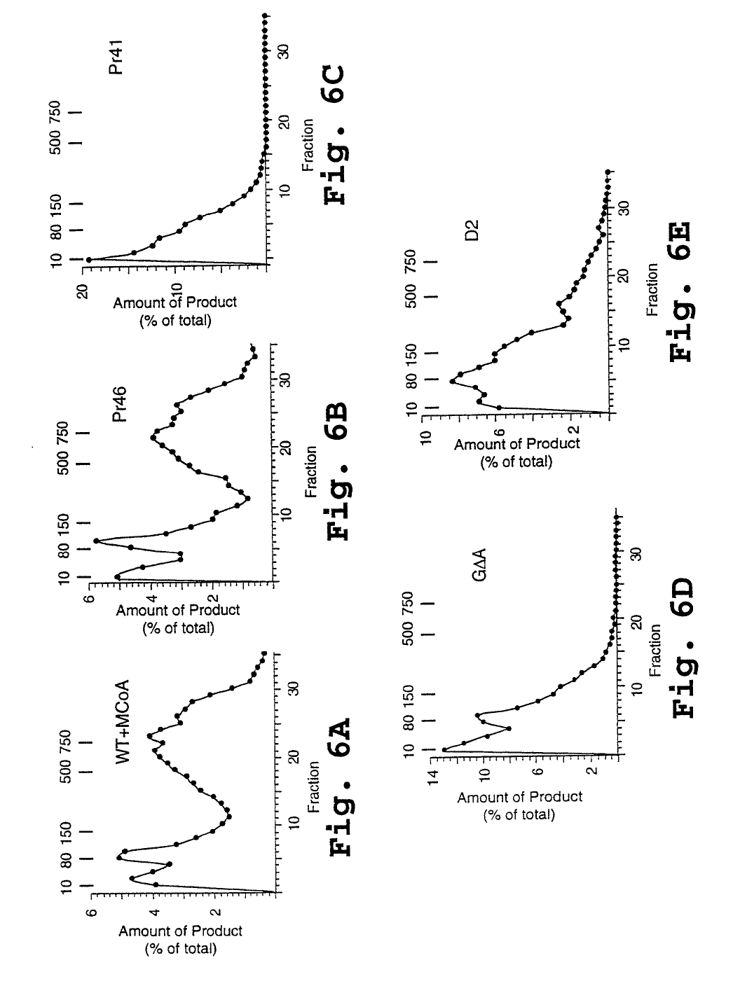

As is shown in FIG. 4B, the Pr41 and GΔA mutants failed to assemble completed capsids, while approximately 40% of the total translation product of both wild-type Gag and the assembly-competent Pr46 mutant assembled into completed capsids. The non-assembling D2 mutant appeared to have generated a small amount of material in the region of completed capsids, but further analysis of this material revealed it to be the trail of a large Gag complex (of approximately 400-500S) that does not comigrate with completed capsids (see FIG. 6E). Thus, like Pr41 and GDA, D2 did not assemble into the 750S completed capsid. Together, these data indicate that the cell-free system appears to reproduce phenotypes of a variety of assembly-defective and assembly-competent mutations in Gag.

### Example 8

### Identification of HIV Capsid Intermediates

The requirement for host factors and ATP suggests that discrete biochemical intermediates exist during the assembly process. Heretofore, such intermediates in HIV capsid assembly have not been described. However, according to a further aspect of the present invention, it is appreciated that the cell-free system of the present invention constitutes a good system for detecting assembly intermediates that would be otherwise difficult or impossible to detect.

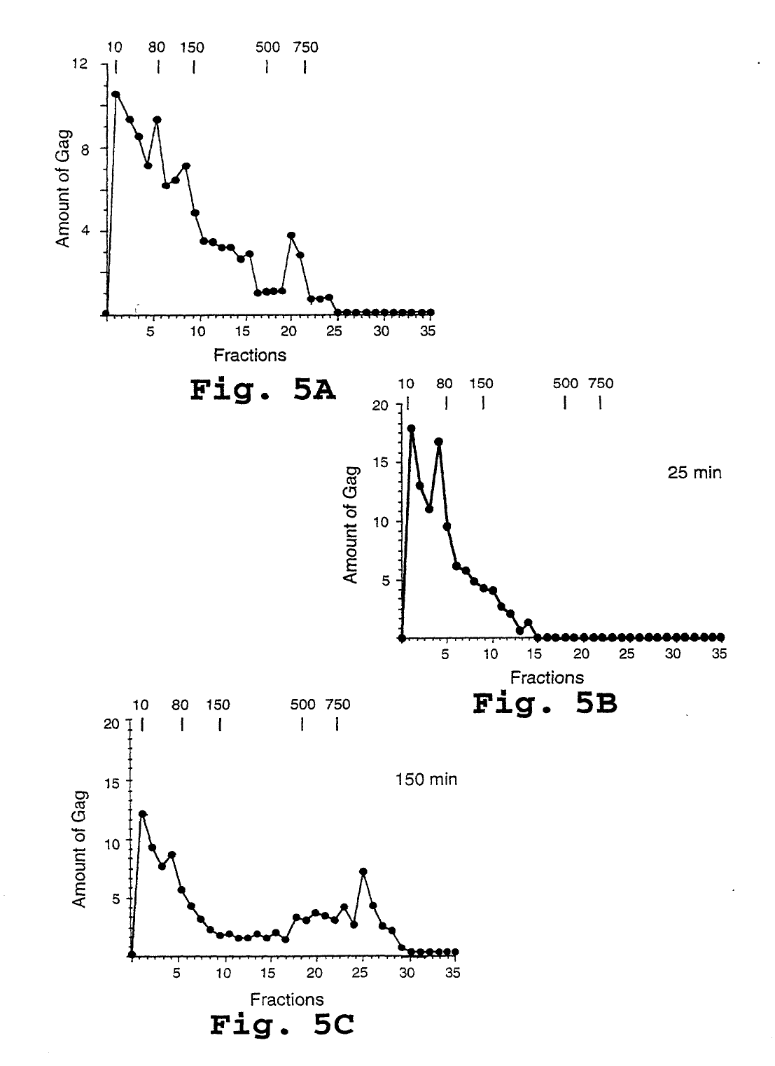

In experiments carried out in support of the present invention, a continuously labeled cell-free reaction was analyzed by velocity sedimentation. Cell-free translation and assembly of Pr55 was performed as described above. Upon completion of the cell-free reaction, the products were diluted into 1% NP40 sample buffer on ice, and were analyzed by velocity sedimentation on 13 ml 15-60% sucrose gradients. Fractions were collected from the top of each gradient, and the amount of radiolabeled Pr55 protein in each fraction was determined and expressed as percent of total Pr55 protein present in the reaction. The calculated positions of 10S, 80S, 150S, 500S, and 750S complexes are indicated with markers above the figures (cf, FIG. 5A). 750S represents the position of authentic immature (de-enveloped) HIV capsids. The intermediate complexes having calculated sedimentation coefficients of 10S, 80S, 150S and 500S are referred to herein as intermediates A, B, C and D, respectively.

Further experiments in support of the present invention indicate that the identified intermediates represent assembly intermediates, as evidenced by the observation that they are present in large quantities at early time points, and are diminished at later times during the reaction. Specifically, pulse-chase analysis was used to follow a small cohort of radiolabeled Pr55 chains over time during the assembly reaction. Cell-free translation and assembly of Pr55 was performed according to the methods set forth in Example 1, except that ³⁵S cysteine was used for radiolabeling. At 4 min into the translation reaction, an excess of unlabeled cysteine was added to the reaction so that no further radiolabeling would occur. Aliquots of the reaction were collected 25 min (FIG. 5C) and 150 min (FIG. 5D) into the reaction. One microliter of each aliquot was analyzed by SDS-PAGE and AR to reveal the total amount of radiolabeled Pr55 translation product (indicated by arrow in FIG. 5B) present at each chase time. The remainder of the aliquots were diluted into 1% NP40 sample buffer on ice, and were analyzed by velocity sedimentation on 13 ml 15-60% sucrose gradients (FIGS. 5C and 5D respectively), in the manner described for FIG. 5A above.

The total amount of radiolabeled Pr55 was the same at 25 min and 150 min into the pulse-chase reaction, indicating that neither further radiolabeling nor degradation of Pr55 chains occurred after 25 min, and confirming that the same population of Pr55 chains was being analyzed at both times.

After 25 minutes of reaction time, all of the radiolabeled Pr55 was found in complexes A, B, and C (FIG. 5C), with no radiolabeled Pr55 chains present in the region of completed 750S capsids. While complexes A and B appear as peaks at approximately the 10S and 80S positions of the gradient, complex C appears as a less distinct shoulder in approximately the 150S position. In marked contrast, examination of the assembly reaction at 150 minutes showed that a significant amount of radiolabeled Pr55 was assembled into completed capsids that migrated in the 750S position (FIG. 5D). Correspondingly, the amount of Pr55 in complexes A, B, and C was diminished by precisely the amount that was now found to be assembled, demonstrating that at least some of the material in complexes A, B, and C constitutes intermediates in the biogenesis of completed 750S capsids.

At extremely short chase times (i.e., 13 min), when only some of the radiolabeled chains have completed synthesis, full length Pr55 chains were found exclusively in complex A on 13 ml sucrose gradients, while nascent chains that are not yet completed were in the form of polysomes of greater than 100S. Thus, polysome-associated nascent chains of Gag constitute the starting material in this pathway, and the 10S complex A, which contains completed Gag chains, is likely to be the first intermediate in the formation of immature capsids. Therefore, complexes B and C may represent later assembly intermediates in the pathway of capsid formation.

As further confirmation that complexes A, B, and C constitute intermediates in HIV capsid assembly, it is shown below that blockade of assembly results in accumulation of Gag chains in the form of complexes with S values corresponding to the S values of A, B and C. Additional evidence is provided by data showing that blockade at different points along the pathway results in accumulation of complexes A, B, and C in various combinations, as determined by the order of their appearance during the course of assembly. For example, if an ordered pathway of intermediates exists, then blockade at early points in the pathway should result in accumulation of one or two Gag-containing complexes corresponding to early putative assembly intermediates, while blockade at a very late point in the pathway would result in accumulation of all the putative assembly intermediates but not the final completed capsid product.

a. Pharmacological Blockade of Assembly

Capsid assembly was disrupted by adding either apyrase post-translationally (as described in Section II. C.4) or detergent co-translationally (as described in Section II.C.2), and the reaction products were analyzed by velocity sedimentation. Material in fractions corresponding to the assembly intermediates and completed capsid were quantified and are presented in Table 1.

TABLE 1

| | A | B/C | Final Capsid |
|---|---|---|---|
| untreated | 2798 | 5046 | 739 |
| +apyrase | 2851 | 5999 | 133 |
| +detergent | 2656 | 6130 | 189 |

The untreated reaction contained Pr55 in complexes A, B, and C, as well as a peak in the final 750S capsid position, while the treated reactions contained no peak at the position of the final capsid product (Table 1). Treatment with either apyrase or detergent resulted in accumulation of additional material in complexes B and C, but did not result in accumulation of additional material in complex A. This is consistent with the idea that complexes B and C are the more immediate precursors of the 750S completed capsids, and that these interventions block the conversion of complexes B and C into the fully assembled capsid end-product.

b. Assembly-Defective Mutants

Further evidence of the existence of assembly intermediates A, B and C comes from experiments carried out in support of the present invention in which the intermediates accumulated when capsid assembly was blocked by specific mutations in Gag. Cell-free reactions were programmed with each of the previously described assembly-competent and assembly-defective Gag mutants (see FIG. 4), and were incubated for 150 min. The reaction products were diluted into 1.0% NP40 sample buffer on ice, and were analyzed by velocity sedimentation on 13 ml 15-60% sucrose gradients then analyzed by velocity sedimentation. Reactions programmed with wild-type Gag (FIG. 6A) or the assembly-competent Pr46 mutant (FIG. 6B) were found to have nearly identical profiles, in which over 30% of the radiolabeled chains synthesized formed completed immature capsids (that migrate at 750S) and the remainder was in the form of residual putative assembly intermediates A and B. Thus, these two assembly-competent forms of Gag appear to be equally efficient at capsid assembly in the cell-free system.

FIG. 6C shows the same analysis for the assembly-defective Pr41 mutant. All radiolabeled chains at the end of the Pr41 cell-free reaction were contained in a single, approximately 10S complex, corresponding to complex A. Since the 10S peak was very large and led to an irregular trail that could be masking 80S or 150S peaks, products of the Pr41 reaction were re-analyzed on a gradient that allowed high resolution in the 1 to 200S size range. All of the Pr41 translation product was in fact present in complex A, which was approximately 10S in size. Thus, in the cell-free system, it appears that Pr41 fails to progress beyond complex A, which is likely to represent the first intermediate in the assembly pathway.

Like Pr41, the myristoylation-incompetent GAA mutant failed to assemble into 750S capsids (FIG. 4B, FIG. 6D), but unlike Pr41, GAA had distinct peaks in both the 10S and 80S regions of the gradient (compare FIG. 6D to FIG. 6C). These data indicate that the GΔA mutant, which contains the entire Gag coding region except for the myristoylation signal, is capable of forming complex A, which appeared to be the first assembly intermediate in the pulse-chase experiment, as well as complex B, but does not progress further towards forming completed capsids. These data suggest that complex B is likely to be the second assembly-intermediate formed in the biogenesis of immature HIV capsids.

As shown above, in the absence of exogenously-added MCoA, wild-type Gag failed to assemble in the cell-free system (FIG. 2A), consistent with previous observations that myristoylation is required for proper capsid assembly to occur. Thus, a cell-free reaction programmed with wild-type Gag but performed in the absence of MCoA would be expected to be blocked at the same point in the assembly pathway as the GΔA mutant. Consistent with this, experiments carried out in support of the present invention demonstrate that assembly performed in the absence of MCoA results in formation of only complexes A and B and therefore closely resembles the GΔA mutant shown in FIG. 6D.

Analysis of a cell-free reaction programmed with the D2 mutant is shown in FIG. 6E. Unlike the previously described assembly-defective mutants, D2 was found to form a spectrum of Gag-containing complexes, including peaks corresponding to complexes A and B (at approximately 10S and 80S), a shoulder corresponding to complex C (in the 150S region), and an additional peak of approximately 400-500S, that will henceforth be referred to as complex D. Note that complex D trails into the 750S region, accounting for the appearance of small amount of assembly in the simpler analysis of capsid formation presented in FIG. 1. However, the detailed analysis presented here makes it clear that in fact there is no discrete peak in the region of completed capsid (750S). Thus, the D2 mutant appears to form a series of complexes corresponding in size to the assembly intermediates seen in the pulse-chase experiment (FIG. 6), as well as an additional complex of larger size, but fails to produce the completed 750S product.

### Example 9

### Host Cell Proteins Involved in Capsid Intermediate Formation

In further experiments carried out in support of the present invention, capsid intermediates formed and isolated as described above were analyzed for the presence of additional protein species. Immunoprecipitation reactions were carried out using several antibodies directed to cellular proteins. Surprisingly, a monoclonal antibody which recognizes a molecular chaperone known as TCP-1, antibody “23 c”, was found to specifically interact with capsid intermediate fractions. TCP-1 is a 55-60 kD polypeptide that resides in a 20S particle and is not known to play a role inviral capsid assembly. Interestingly, antibody 23 c does not recognize the human or yeast homologs of TCP-1, but it does recognize a number of other eukaryotic proteins, presumably through recognition of their common C-terminal epitopes (LDD-COOH).

Further experiments in support of the invention revealed that the 23c reactive protein present in wheat germ extract migrates on SDS polyacrylamide gels as a 68 kilodalton protein. Further analysis reveals that the protein includes a peptide region having the following sequence: PRPYLDVKQRLKAARVIRSLLRSN (SEQ ID NO: 2) and has the full open reading frame of SEQ ID NO: 5.

Association of HP68 with the previously identified capsid assembly intermediates was assessed by measuring immunoreactivity of the 23c antibody. In these experiments, cell-free capsid formation reactions were programmed with Gag transcript (Example 1), pulse-labeled with 35-S cysteine for 3 minutes, and then chased with an excess of unlabeled cysteine. Under these conditions, chains synthesized during the first 25 minutes of the reaction are radiolabeled, while subsequently formed chains are unlabeled. Aliquots of the cell-free reaction were removed at various times during incubation and were either analyzed directly by SDS-PAGE or were subjected to immunoprecipitation with 23 c antibody (Example 9).

In these reactions, it was verified that the total number of radiolabeled chains synthesized over time remained relatively constant, while the number of radiolabeled chains in the form of fully assembled capsids increased progressively over the course of reaction. from 1.0% to 50.0%, with the largest increase in completed capsids occurring after 75 minutes. In contrast, the number of radiolabeled Gag chains bound to HP68 (as assessed by immunoprecipitation with 23 c) was very low just after synthesis was completed, but increased significantly over time, reaching a peak at approximately 75 minutes into the incubation, then decreasing substantially during the final hour of the cell-free reaction. These observations are consistent with the conclusion, illustrated below, that HP68 does not bind specifically to either newly-synthesized, unassembled Gag chains or to fully-assembled capsids.

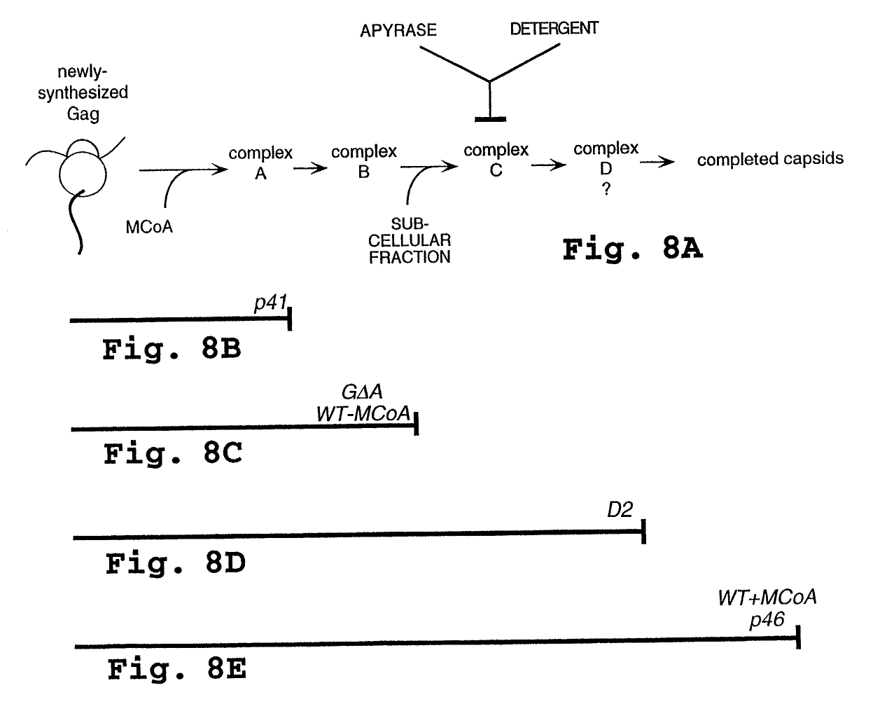

In further experiments, radioactive HIV assembly intermediates formed as described above were subjected to velocity sedimentation, followed by immunoprecipitation using the 23 c antibody. With reference to the schematic shown in FIG. 8A, radiolabeled Gag chains in the form of the 80S and 500S assembly intermediates (intermediates B and D, respectively) were immunoreactive with 23c antibody, while fully assembled 750S capsids were not immunoreactive. Although intermediate C (150S) showed little or no immunoreactivity in these experiments, there is also very little of this intermediate present in the mixture at the time point assayed (2 hours), so the presence of HP68 in this fraction cannot be ruled out.

These results were also confirmed using assembly incompetent mutant viruses, as discussed above. Table 2 shows the results of experiments in which various assembly incompetent mutants or reaction manipulations were used to assess HP68 association with the above-defined intermediates. Cell-free reactions were programmed with wild-type (“Gag”), mutants Pr46 (“p46”), GΔA or Pr41 (“p41”), or were carried out in the presence of detergent (“Gag +det”) or with the addition of apyrase (“Gag +apy”). Distribution of the above-described intermediates A-D and completed capsids was assessed for each condition, as described above, and 23c immunoreactivity was determined.

TABLE 2
Distribution of Gag-containing Intermediates

| | | | | | Complete | 23c immuno- |
|---|---|---|---|---|---|---|
| | A | B | C | D | capsid | reactivity |
| Gag | + | ++ | + | ++ | +++ | ++ |
| p⁴⁶ | + | ++ | + | ++ | +++ | ++ |
| Gag + det | ++ | ++ | + | − | − | + |
| Gag + apy | ++ | ++ | + | − | − | + |
| GΔA | ++ | ++ | − | − | − | + |
| p41 | +++ | − | − | − | − | − |

As illustrated, the absence of 23c immunoreactivity in the Pr41mutant reaction, which fails to form any high molecular weight intermediates, indicates that there is no association of HP68 with intermediate A; in contrast, wild-type Gag and Pr46 mutant, which form high intermediates B-D are highly reactive. In the presence of detergent or apyrase, assembly intermediates A-C accumulate, as described above; under these conditions, 23c immunoreactivity was observed.

The foregoing data support one of the discoveries of the present invention that assembly of HIV capsids involves a host protein derived from the host cell, exemplified herein by HP68. In accordance with the present invention, HP68 is (i) is immunoreactive with monoclonal antibody 23c, and (ii) includes the sequences SEQ ID NO: 2. Specifically WGHP68 is one such homologue and is represented as SEQ ID NO: 5. The present invention also appreciates that other cellular homologs of HP68 perform a similar function in hosting HIV assembly. Specifically contemplated by the present invention is a human homologue of HP68, which is associated with intermediates B-D present in human cell systems. By “homologue” is meant a protein or proteins that resemble HP68 in sequence (at least about 60% sequence identity by a standard protein/nucleotide sequence comparison algorithm), and which can be isolated from or detected in association with HIV capsid intermediates B-D.

### Example 10

### Correspondence of Cell-Free Capsid Intermediates to Cell-Produced Capsid Intermediates

Cos-1 cells were transfected with a transfection vector encoding Pr55 cDNA, as described in the Examples. Four days later, the medium from the cells was collected. Viral particles in the medium were harvested by ultracentrifugation through a 20% sucrose cushion and then treated with detergent to remove envelopes. The transfected cells were solubilized in detergent to generate the cell lysate. The particles from the medium (FIG. 6A, right ordinate, open circles) and the detergent lysate of the cells (FIG. 6A, left ordinate, closed circles) were analyzed in parallel by velocity sedimentation on 13 ml 15-60% sucrose gradients. The amount of Pr55 protein in each fraction of these gradients was determined by immunoblotting and is expressed as percent of total Pr55 protein present. The calculated positions of 10S, 80S, 150S, 500S, and 750S complexes are indicated with markers above each graph. 750S represents the position of authentic immature (de-enveloped) HIV capsids.

Different cultures of Cos-1 cells were transfected with a transfection vector encoding the Pr41 mutant (FIG. 6B) or the D2 mutant (FIG. 6C). Transfected cells were lysed in detergent, and the lysate was analyzed by velocity sedimentation on 13 ml sucrose gradients, as in the experiments described with reference to FIG. 8A, above. The amount of capsid protein in each fraction of these gradients was determined by immunoblotting with anti-Gag antibody, and was expressed as percent of total immunoreactive protein present in each reaction. As shown, a substantial amount of fully assembled 750S capsid was present in the medium (FIG. 6A, open circles), while the cell lysate contained no 750S capsids (FIG. 6A, closed circles). These data are consistent with correspondence of intermediates in vivo with those reported above for cell-free capsid synthesis and assembly.

Analysis of the Pr41 mutant transcript is shown in FIG. 6B. This mutant appears to be blocked after the first assembly intermediate in the cell-free system. Analysis of the D2 mutant, which appears to be blocked at the end of the assembly pathway in the cell-free system, shows accumulation of corresponding Gag-containing complexes within cells. Cos cells were transfected with each of these mutants, and the medium as well as the lysate were examined by immunoblotting. Medium from cells transfected with the assembly-defective Pr41 or D2 mutants did not contain 750S completed capsids. The cell lysate of Cos cells transfected with the Pr41 mutant contained only material that peaked in the 10-S region of the velocity gradient (FIG. 6B), resembling what had been found when the Pr41 mutant was expressed in the cell-free system (see FIG. 5C). The observation that the Pr41 reaction product migrated as a single complex that peaked in the 10S region was confirmed by analysis on a variety of different velocity sedimentation gradients that allowed higher resolution in the 1 to 200S size range.

In contrast, the cell lysate of Cos cells transfected with the D2 mutant contained a spectrum of immunoreactive complexes that ranged in size from 10-S to 500-S (FIG. 6C), resembling what was found when D2 was expressed in the cell-free system (FIG. 5E). Thus, the data from transfected cells suggests that the behavior of Gag mutants in the cell-free system reflect events in capsid assembly that occur in living cells.

### Example 11

### Model for Capsid Assembly

A model of the HIV capsid assembly pathway is shown in FIG. 8A. This model is based on the simplest interpretation of the data presented herein. This model is presented for purposes of summarizing these data, and is not to be construed as a representation of a particular underlying mechanism to which the present invention must adhere. In particular, the exact relationship of the subcellular fraction dependent step, as well as the apyrase- and detergent-sensitive steps to the pathway are not to be taken as a basis for limiting the claimed method or cell-free system of the present invention. Moreover, although the order of complex formation shown is consistent with the data presented, this order should not be used to limit the claimed intermediate compositions.

According to the model presented in FIG. 8A, newly-synthesized Gag proteins are myristoylated co-translationally. Nascent Gag polypeptides appear to chase into completed immature capsids by way of a series of Gag-containing complexes (complexes A, B, C, and D). Evidence from the studies reported herein suggests that complexes A, B, and C may constitute assembly intermediates. Complex D may similarly constitute an assembly intermediate or may represent a side-reaction. A subcellular, detergent-resistant factor appears to be required for capsid formation. In addition, ATP and a membrane fraction are also required for assembly to take place, as evidenced by apyrase and detergent sensitivity of the assembly process.

FIGS. 8(B-D) show the proposed correspondence between assembly mutants p41, GΔA, D2 and p46 to the model pathway, based on the data presented above.

### Example 12

### HP68 is Essential for HIV-1 Capsid Formation

1. Purification and sequencing of HP68

For immunoaffinity purification, 1 ml WG extract was centrifuged at 100,000 rpm in a Beckman TL100.2 rotor for 15 min. The supernatant was subjected to immunoprecipitation using 50 μg of affinity purified 23c antibody (Stressgen) or an equivalent amount of control antibody (α-HSP 70, Affinity Reagents). Immunoprecipitation eluates were separated by SDS-PAGE and transferred to a polyvinylidene difluoride membrane. A single 68 kD band was observed by Coomassie-staining in the 23c immunoprepicipation lane but not on the column. A portion of this band was excised for microsequencing (ProSeq, Salem, Mass.) and the remainder was used for immunoblotting to confirm that the band was recognized by the 23c antibody. The purified protein, which was blocked at the N-terminus, was cleaved with CNBr and treated with o-phthalaldehyde to allow selective microsequencing using Edman degeneration of peptides containing proline near the N-terminus.

2. cDNA amplification

The following degenerate 3′ oligonucleotides corresponding to the C-terminal peptide sequence of WGHP68 3′ was synthesized: ATGAATTC(ACTG)GG(ACTG)CG(GA)TA(GA)TT(ACTG)GT(ACTG)GG(GA)TC (SEQ ID NO. 3) and ATGAATTC(ACTG)GG(CT)CT(GA)TA(GA)TT(ACTG)GT(ACTG)GG(GA)TC (SEQ ID NO. 4). The WGHP68 coding region was amplified by PCR using WG cDNA (Invitrogen), as the template, 3′ oligos corresponding to the WGHP68 C-terminal peptide sequence and 5′ oligos corresponding to the vector into which the cDNA was cloned. This PCR reaction was performed four independent times and each time yielded a single 2 kB product. These PCR products were ligated into vectors by TA cloning (Invitrogen). DNA sequencing revealed each cDNA product to be identical. 3′ and 5′ coding and non-coding ends were obtained through nested RACE PCR reactions using degenerate oligos corresponding to sequences in the internal region of HP28. From overlapping cDNA clones, a complete open-reading frame for WGHP68 was defined. The start was identified by the presence of a defined Kozak consensus sequence at the initiating methionine, the presence of two in-frame stop codons upstream of the first methionine, the absence of ATG codons upstream from the presumptive start site (Kozak, Mamm Genome (1996) 7:563-74), and by homology to the human homologue in GenBank (Bisbal et al. J Biol Chem, (1995) 270:13308-17). The coding sequence for WGHP68 (SEQ ID NO: 5) has been deposited in GenBank under accession number AY059462.

3. Generation of Antisera

Polyclonal rabbit antisera were generated against C-terminal peptides of Hu and WGHP68 (FIG. 9) and against the 19 N-terminal amino acids of human RNase L by injecting rabbits with peptides coupled to KLH. Affinity-purified αHuHP68b antisera was prepared by binding antisera to the HuHP68 C-terminal peptide coupled to agarose and eluting with glycine.

4. Transfections, Immunoprecipitation, Immunofluorescence, and Immunoblotting

Cos-1 cells were transfected using Gag expression plasmids pCMVRev and PSVGagRRE-R described in Simon et al, J. Virology, (1997) 71:1013-18. HP68 plasmids for mammalian expression were constructed by using PCR to insert the coding regions for WGHP68, amino acids 1-378, Nhel/Xbal of pCDNA 3.1 (Invitrogen). Coding regions of all constructs were sequenced. Cells were transfected using Gibco Lipofectamine (Cos-1) or Lipofectamine Plus (293T). All transfections used a constant amount of DNA (18 μg per 60 mm dish). Medium was changed 24 hours after transfection and harvest was performed 28 or 60 hours after transfection for immunofluorescence and immunoblotting respectively. For immunofluorescence, cells were fixed in paraformaldehyde, permeabilized with 1% triton, and incubated with mouse HIV-1 Gag antibody (1:50) and affinity-purified HuHP68 antiserum (1:2000), followed by Cy3- and Cy 2-coupled secondary (Jackson) (1:200). 178 cells were quantitated. For immunoblotting in FIG. 12 rat IgG was added to medium as a tracer at 10 μg/ml at the time of harvest, and cells were harvested in SDS sample buffer with boiling. For quantitation of immunoblotts, bands were compared to an immunoblot standard curve generated with known quantities of sample.

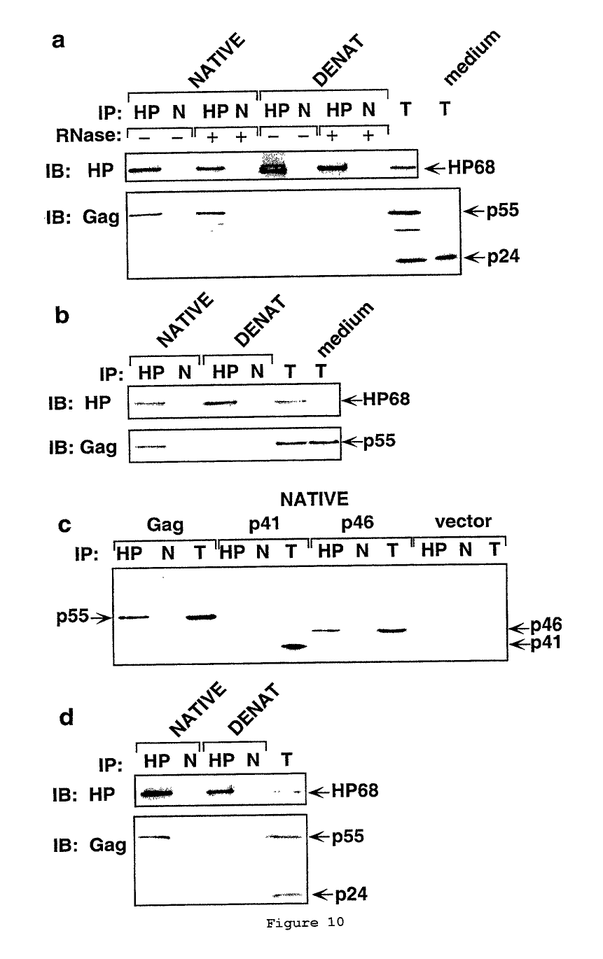

For immunoprecipitations followed by immunoblotting (FIGS. 10 and 14), affinity purified α-HuHP68 antisera described above was coupled to Protein A beads (7mg/ml beads) to generate αHuHP68b. Confluent Cos-1 cells in 60mm dish were transfected, harvested in 300 μl NP40 buffer and 100 μl of lysine was immunprecipitated with 50 μl of αHuHP68b. Immunoprecipitates were analyzed by SDS-PAGE followed by immunoblotting with antibodies described.

5. Immunodepletion-reconstitution

WG extract (150 μl) was immunodepleted for 45 min at 4° C. with 100 μl beads coupled to antibody against WGHP68. Cell-free reactions (15 μu) were programmed (Lingappa et al., J. Cell Biol. 136:567-81 (1997)) using non-depleted WG or depleted WG. To some reactions containing depleted WG, purified WGHP68-GST or HuHP68-GST fusion protein or GST alone was added (2 μl of approx. 20 ng/μl) at the start of the reaction. After 3 hours at 26° C., NP40 was added to a final concentration of 1% and reactions underwent velocity sedimentation (5 ml, 15-60% sucrose gradients, Beckman MLS55 rotor: 45,000 rpm, 45 min). Thirty fractions, collected using a fractionator, were analyzed by SDS-PAGE and AR, followed by densitometry of Gag in each lane. For Proteinase K digestion, aliquots of fractions from the 500S and 750S regions of the gradient were collected and subjected to a 10 min incubation at RT with either no Proteinase K or 0.1 μg/ml Proteinase K. Digestion was terminated by adding SDS and freezing. Samples were analyzed by SDS-PAGE and AR. Graphs show average of three independent experiments (+/− SEM).

To generate purified HP68, WGHP68 and HuHP68 were subcloned into a pGEX vector (Pharmacia), to encode fusion proteins containing GST at the N-terminus. Expression was induced with 1 mM IPTG for 3 hours; sarcosyl (0.5%) and PMSF (0.75 mM) was added after sonication. 17,000×g supernatent was incubated with glutathione beads and eluted with 40 mM glutathione in 50 mM Tris, pH 8.0. Concentration of fusion protein and GST in eluate was determined using the Coomassie Plus protein assay (Pierce).

Two cell-free reactions were programmed with HIV-1 Gag transcript and immunodepleted WG, and WGHP68-GST was added to one of these reactions. In parallel, Cos-1 cells were transfected resulting in expression of Gag and release of immature HIV- 1 particles. The cell-free reactions and medium from transfected cells was treated with 1% NP40 to remove envelopes, and membranes associated with capsids, subjected to velocity sedimentation on 2ml 20-66% sucrose gradients (Beckman TLS55 rotor, 35 min, 45,000 rpm).

### Example 12

### Conformer of HP68 Essential for Assembly of HIV-1 Capsids

1. Wheatgerm HP68 (WGBP68) was isolated from WG extracts by immunoaffinity purification using 23c antibody. Microsequencing yielded two well-defined sequences of 24 or more amino acids. Each sequence was approximately 70% homologous to a different region of a single 68 kD protein identified as human RNase L inhibitor (Bisbal et al. JBC (1995) 270:13308-17; GenBank A57017, SEQ ID NO: 6) (FIG. 9). Using degenerate oligonucleotides (SEQ ID NOS: 3 and 4) corresponding to the C-terminal peptide, a 2 kB cDNA was amplified from a WG cDNA mixture. Sequencing revealed that this cDNA has 70% identity overall to the cDNA coding for the 68 kD human RNase L inhibitor (here termed HuHP68) (Bisbal et al. JBC (1995) 270:13308-17; Bisbal et al. Methods Mol Biol (2001) 160:183-98). The open reading frame WGHP68 was deduced and its full amino acid sequence was predicted (FIG. 9). The 604 amino acid sequence of WGHP68 shows 71% identity overall with the 599 amino acid sequence but of human RNAse L inhibitor (HuHP68). Both WGHP68 and HuHP68 contain two canonical ATP/GTP-binding motifs (Traut T. Eur J. Biochem (1994) 222:9-19) as well as the LDD-_{COOH} epitope (FIG. 9).

HuHP68 is known to bind and inhibit RNase L (Bisbal et al. JBC (1995) 270:13308-17; Bisbal et al. Methods Mol Biol (2001) 160:183-98), an interferon-dependent nuclease associated with polysomes (Salehzada. et al JBC (1991) 266:5808-13; Zhou et al. Cell (1993) 10 72:753-65) and activated by the interferon-sensitive 2′-5′ linked oligoadenylate (2-5S) pathway. Interferon-dependent induction and activation of RNase L results in degradation of many viral RNAs (Player et al. Pharmacol Ther. (1998) 78:55-113; Samuel C. Virology (1991) 183:1-11; Sen et al. JBC (1992)267:5017-20). Previously, overexpression of the 68 kD RNAse L inhibitor (HuBP68) in HIV-1-infected cells has been shown to increase virion production by reducing RNase L activity, resulting in higher levels of HIV-1 RNA and HIV-1-specific protein (Martinand et al. J. Virology (1999) 73:290-6). These findings that WGHP68 binds to Gag-containing, post-translational intermediates during cell-free HIV-1 capsid assembly led to further investigation of whether HuHP68 binds to and acts on fully-synthesized Gag chains post-translationally in cells, in addition to binding and inhibiting RNase L as previously described (Salehzada et al. JBC (1991) 266:5808-13; Zhou et al. Cell (1993) 72:753-65).

2. Association of HP68 with HIV-1 Gag infected Human Cells

To analyze the function of HP68 in cells, a peptide-specific polyclonal antibody was generated against both C-terminal residues of WGHP68, and C-terminal residues of HuHP68 (FIG. 9). These antisera specifically recognize a 68 kD protein in WG and in primate cells respectively, by immunoporecipitation as well as Western blotting. To determine whether HP68 is associated with assembling HIV-1 Gag chains in human cells, human 293T cells were transfected with the pBRUΔenv plasmid. Immunoprecipitates were analyzed by Western blotting using a monoclonal antibody to HIV-1 Gag. Hiv-1 Gag is co-immunoprecipitated by αHuHP68 under native condition but not after denaturation (FIG. 10A). HP68 appears to associate with Gag post-translationally. These data reveal the HuHP68 is associated with HIV-1 Gag in human cells that are producing mature HIV-1 virions.

3. HP68 associated with Gag post-translationally in human cells

Further investigation revealed that HP68 is associated with Gag in RNase-treated and unteated cell lysates analyzed in parallel (FIG. 10A). These findings that HuBP68 binds completely-synthesized Gag chains, and does so in the absence of intact RNA, indicates that this host protein is bound to Gag-containing complexes post-translationally. FIG. 10B demonstrates that Gag is associated with HP68 under native conditions, but not after denaturation when immunopercipitated with αHuHP68b. This confirms that HP68 binds HIV-1 Gag in the absence of the HIV-1 protease and other HIV-1 specific proteins. FIG. 10C demonstrates that HP68 is associated with wild-type Gag and with the assembly-competent p46 mutant, but is not associated with assembly incompetent p41 mutant. Thus, HP68 appears to associate specifically with assembling Gag chains in mammalian cells, as it did in the cell-free system. Confirmation studies were performed with fully infectious human T-cells, wherein immunoprecipitation was performed with αHuHP68 demonstrating that HP68 associates with Gag in infected human T-cells. FIG. 4D shows that αHuHP68b co-immunopreciptated HP68 and Gag from T-cell lysates. Confirmation of co-association of HP68 and Gag was demonstrated with immunofluroescent microscopy (FIG. 11). HP68 staining reveals two different patterns of localization. HP68 is present in a diffuse pattern in 100% of the cells that fail to become transfected and do not express HIV Gag (two cells on left in FIGS. 11A-C), as well in 100% of control cells that are transfected with constructs expressing control proteins. In cells expressing HIV Gag, HP68 is found in a coarsley clustered pattern (FIGS. 11D and F) FIGS. 11C, F and I show a merged image where there is a striking co-localizaion of HP68 and Gag in the yellow coarse cluster. Recruitment of HP68 into clusters containg Gag is seen in 100% of cells expressing HIV-1 Gag. In contrast, when cells are transfected with pBRUp41Δenv, which encodes an assembly defective mutant, HP68 is not found in a clustered pattern or co-locaalized with HIV Gag (FIGS. 11G-I).

4. HP68 Mutant Binds HIV-1 Gag and Blocks HIV-1 Particle Formation

To examine HP68 function (i.e. is HP68 association with Gag important for HIV-1 particle formation), Cos-1 cells were co-transfected with WGHP68-Trl and a Gag expression plasmid (FIG. 12). Increasing expression of WGHP68Tr-1 results in a 4.7 fold dose-dependent decrease in the amount of HIV-1 Gag protein in the medium (FIG. 12A, p55 blot and graph). Gag and actin levels in cell lysates remained unchanged (FIG. 12B), indicating that the effect of WGHP68-Tr1 is not mediated by changes in Gag synthesis or degradation, and that WGHP68-Tr1 is not toxic to cells. Reduction in virion formation upon WGHP68-Tr1 expression, even when Gag levels are unchanged, suggests that HP68 promotes virion formation by a post-translational mechanism. Co-immunoprecipitation of Gag with epitope-tagged WGHP68-Tr1 confirmed that WGHP68-Tr1 competes with wild-type HP68 for binding to HIV-1 Gag (data not shown).

5. Capsid Assembly is Inhibited by Depletion of HP68 and Restored by Reconstitution.

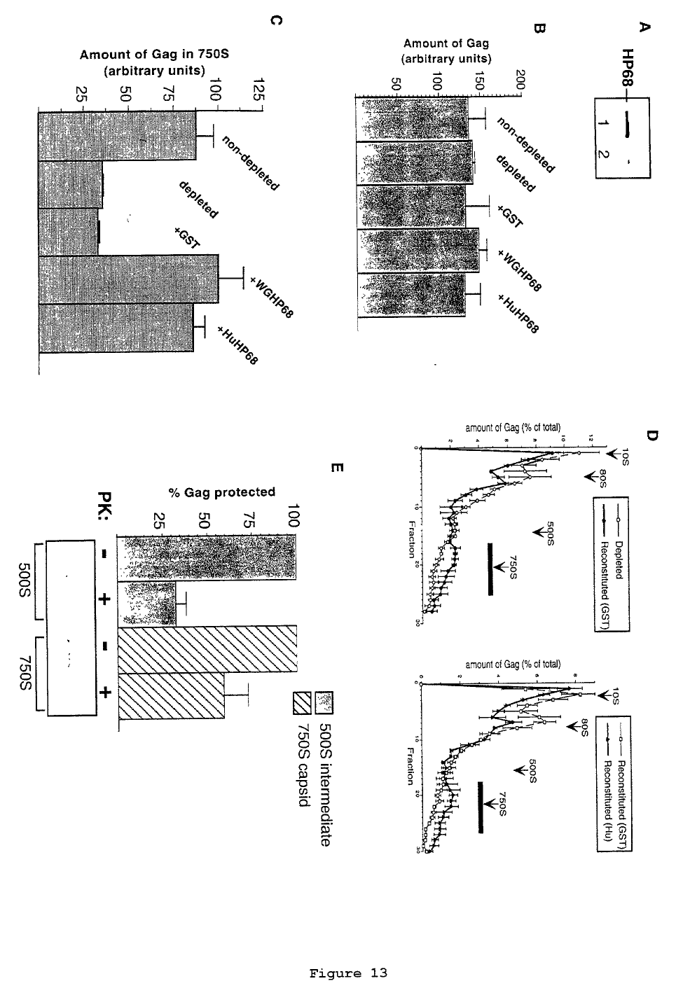

To demonstrate that HP68 is essential for post-translationally events in immature capsid assembly, endogenous HP68 was immunodelepted with αWGHP68 from wheat germ extracts prior to programming for a cell free reaction. FIG. 13A shows in lane 1 vs 2 an extract with reduced levels of WGHP68, but which can still support the same amount of Gag production (FIG. 13B, non-depleted vs. depleted). FIGS. 13C and D show that when immunodepleted (for HP68) cell free extracts are programmed with HIV-1 Gag transcript that the 750S completed capsid were dramatically reduced. Furthermore, the depleted reaction appeared to be arrested at the 500S post-translational assembly intermediate complex, with accumulation of other previously-identified post-translational assembly intermediates (10S and 80S), but no 750S completed immature capsid product (FIG. 13D).

Reconstitution was demonstrated by the addition of either WGHP68-GST or HuHP68-GST to HP68 immunodepleted WG extraxt programmed with HIV-1 Gag transcript, a 3-fold increase in the amount of 750S capsid was observed (FIGS. 13C and D). This is a level observed in non-depleted extract. Addition of either fusion protein (WGHP68-GST or HuHP68-GST) had no effect on the total amount of radiolabele Gag synthesized (FIG. 13B), indicating that the reconstituted protein acts post-translationally. These findings demonstrate that HP68 is required for conversion of post-translational assembly intermediates into completely assembled 750S immature HIV-1 capsids in the cell-free system. In addition, further experiments demonstrate that HP68 promotes a conformational change in capsid structure, converting protease-sensitive capsid assembly intermediates into immature capsid structures that are relatively resistant to exogenous proteases. FIG. 13E shows that upon treatment of protease K to the sucrose gradient fractions, 500S and 750S, that 500S capsid assemby intermediates were sensitive to protease digestion while 750S completed capsid were relatively protease resistant. Thus, the 750S capsid has undergone a conformational change, with the help of HP68, which prevents exogenous proteases from degrading the completed capsid.

6. HP68 Selectively Associates with HIV-1 Gag and Vif but not with RNase L

FIG. 14 shows that the HP68 protein that facilitates HIV-1 capsid formation binds HIV-1 Gag and Vif proteins but does not bind RNase L in human cells, which have been transfected with plasmids expressing Gag alone or with the plasmid pBRUΔenv. These findings suggest that HP68 not only acts by two different mechanisms but resides in two different complexes in host cells as well. In one complex, HP68 associates with and inhibits RNase L, a cellular protein that is upregulated by interferon, binds to ribosomes, and promotes degradation of viral RNA (Zhou et al. Cell (1993) 72:753-65; Player et al. Pharmacol Ther (1998) 78:55-113; Samuel C. Virology (1991) 183:1-11; Sen et al. JBC (1992) 267:5017-20). An aspect of this invention is that, as described above, HP68 is also present in a second, separate complex (assembly intermediate), in which HP68 acts post-translationally to promote virion formation.

To demonstrate these differences in HP68 and the specificity for HIV proteins, Cos-1 cells expressing pBRUΔenv were subjected to immunoprecipitation using αHuHP68b followed by immunoblotting with antibodies to Gag, Vif, Nef, RNase L and actin. αHuHP68b co-immunoprecipitated Gag and Vif under native conditions but not denatured conditions. RNase L and HIV Nef protein were not co-immunoprecipitated, indicating that HP68 is associated with select HIV proteins in a complex that does not contain RNase L.

Thus, the identification of HT68, acting specifically to facilitates HIV capsid, is a specific drug target for blocking the production of HIV virions. Specifically blocking only the conformer involved in HIV production and not a different conformer that may be involved in necessary cell function is important for whose depiction may lead to a different disease state, eg. chronic fatigue syntrome. While in this case the two demonstrated functions of HP68 both promote viral replication, one specifically for HIV the other for viruses in general, HP68 is sure to have, as yet unknown, necessary functions for the host cell that is not infected with viral particles. An important aspect of this invention is identifying conformers specifically involved in viral replication and identifying drugs that block their activity.

All patent and literature references cited herein are incorporated herein in their entireties.

While the invention has been described with reference to specific methods and embodiments, it will be appreciated that various modifications and changes may be made without departing from the invention.

What is claimed is:

1. A method of isolating an HIV capsid intermediate, said method comprising the steps of:
combining HIV Gag Pr55 mRNA with a cell-free protein translation mixture containing myristoyl coenzyme A;
incubating said translation mixture for a period of time sufficient to assemble Gag Pr55 mRNA translation products into immature HIV capsids;
identifying said capsid intermediates on a linear sucrose gradient; and
isolating said capsid intermediates by immunoprecipitation with antibodies specific for HIV Gag, whereby isolated capsid intermediates are obtained.

2. The method according to claim 1, wherein the amount of said myristoyl coenzyme A is about 0.1 to 100 micromolar.

3. The method according to claim 1, wherein said cell-free extract contains a detergent sensitive fraction from eukaryotic cells.

4. The method according to claim 1, wherein said cell-free extract contains a detergent-insensitive fraction from eukaryotic cells.

5. The method according to claim 1, wherein said cell-free extract contains an ATP sensitive fraction from eukaryotic cells.

6. A method of isolating an HIV capsid intermediate, said method comprising the steps of:
combining HIV Gag Pr55 mRNA with a cell-free protein translation mixture containing myristol coenzyme A present in a concentration ranging from about 0. 1 to 100 micromolar, wherein said cell free mixture contains a detergent-insensiteve or a detergent sensitive fraction from eukaroytic cells;
incubating said translation mixture for a period of time sufficient to assemble Gag Pr55 mRNA translation products into immature HIV capsids;
identifying said capsid intermediates on a linear sucrose gradient; and
isolating said capsid intermediates by immunoprecipitation with antibodies specific for HIV Gag, whereby isolated capsid intermediates are obtained.

7. A method of identifying chaperone proteins involved in HIV capsid assembly, said method comprising the steps of:
denaturing affinity purified capsid intermediate complexes so that said complexes are separated into chaperone proteins and capsid proteins;
removing separated capsid proteins with monoclonal antibodies specific for capsid proteins leaving a mixture of chaperone proteins;
isolating individual chaperone proteins from said mixture; sequencing said individual chaperone proteins, and
comparing the sequences of said individual chaperone proteins to known sequences of host proteins, whereby the identity of host proteins that are involved in HIV capsid assembly are obtained.

8. A method of identifying chaperone proteins bound to HIV capsid intermediates produced in a mammalian cell, said method comprising the steps of:
sequencing a human homologue to said chaperone protein identified in the cell-free system according to claim 7 using degenerate primers;
expressing said human homologue in the cell-free system that has been immunodepleted for cell-free chaperone proteins wherein said sequence of human homologue is cloned into an expression vector; and
measuring capsid formation in a cell-free translation system programmed with HIV Gag compared to a cell-free translation system programmed with HIV Gag that has not been immunodepleted, whereby comparable amounts of HIV capsid formation identifies chaperone protein produced in mammalian cells that bind to HIV capsid intermediates.

9. A method of identifying chaperone proteins bound to HIV capsid intermediates produced in a mammalian cell, said method comprising the steps of:
sequencing a human homologue to said chaperone protein identified in the cell-free system according to claim 7 using degenerate primers;
expressing said human homologue in HIV infected mammalian cells that are stably transfected with a dominant negative HP68 mutant wherein said sequence of human homologue is cloned into an expression vector; and
measuring HIV viral release from said cells compared to cells not stabely stransfected with a dominant negative HP68 mutant, whereby restoraion of HIV viral release identifies chaperone proteins produced in mammalian cells that bind to HIV capsid intermediates.

10. A method of identifying conformers of host chaperone proteins that bind to HIV capsid intermediates, said method comprising the steps of:
contacting isolated host proteins having an amino acid sequence substantially similar to a host chaperone protein with a plurality of monoclonal antibodies that bind to said host protein;
identifying from said plurality of monoclonal antibodies those that bind to a subset of said host proteins and do not bind to said host chaperone proteins;
isolating said host cell proteins so identified; and
determining whether said conformer functions to facilitate assembly of HIV capsid intermediates, whereby conformers that so function are identified as conformers of said host chaperone protein.

11. A method of identifying a functional HP68 conformer, said method comprising the steps of:
(a) isolating an RNase L inhibitor in cells not infected with HIV that does not bind to HIV Gag;
(b) isolating HP68 that binds HIV Gag in cells producing HIV Gag;
(c) expressing said RNase L inhibitor in a cell-free translation system that has been immunodepleted for HP68, is programmed with HIV Gag mRNA and comprises an expression vector comprising a nucleic acid sequence encoding said RNase L inhibitor;
(d) expressing said HP68 in a cell-free translation system that has been immunodepleted for HP68, is programmed with HIV Gag mRNA and comprises an expression vector comprising a nucleic acid sequence encoding said HP68; and
(e) comparing capsid formation in step (c) to capsid formation in step (d), whereby a difference in amount of HIV capsids produced is indicative of a conformer of HP68 specific for HIV capsid formation.

12. A method of producing monoclonal antibodies to a conformer of a host protein that is involved in assembly of immature HIV capsids, said method comprising the steps of:
immunizing knockout mice with a host chaperone protein, wherein said knockout mice have a non-functional gene that no longer codes for said conformer and lack the ability to produce said protein;
producing hybridoma cells from spleens of said mice;
screening said hybridoma cells for production of antibodies to both a native and a denatured conformer of said host chaperone protein; and
propagating hybridoma cells producing antibodies that bind substantially specifically to said host chaperone protein and not to conformers of said host chaperone protein that do not bind Gag and do not facilitate HIV capsid assembly, whereby antibodies to native and denatured said protein or peptide conformer of interest are produced.

13. Monoclonal antibodies produced according to the method of claim 12.

14. Binding fragments to said conformer derived from monoclonal antibodies produced according to the method of claim 12.

15. A method of identifying a binding site between a host chaperone protein and an intermediate in HIV capsid assembly, said method comprising the steps of:
obtaining a conformational epitope map of said host protein using monoclonal antibodies produced according to the method claim 12;
obtaining a conformational epitope map of HIV Gag using monoclonal antibodies specific for Gag;
obtaining a conformational epitope map of HIV capsid intermediates using monoclonal antibodies specific for HIV Gag and antibodies produced according to the method of claim 12; and
comparing conformational epitope maps of HIV Gag, capsid intermediate complex and said conformer, whereby the binding site on said conformer for HIV Gag is identified and the binding site on HIV Gag for said conformer is isdentified.

16. A method for identifying compounds that interfere with HIV capsid assembly by specifically binding to and preventing said conformer from binding to HIV Gag, said method comprising the steps of:
(a) screening databases for compounds that bind to the binding site identified according to the method of claim 15, whereby potential compounds are obtained;
(c) screening said potential compounds for test compounds that bind substantially specifically to a host chaperone protein for HIV capsid assembly but not to conformers of said host chaperone proteins that do not bind HIV Gag;
(d) screening said test compounds in a cell free translation system, wherein efficacy is measured by a decrease in HIV capsid production; and
(f) further screening said compounds for ability to block HIV capsid formation in mammalian cells infected with HIV, whereby compounds that block HIV capsid formation are identified.

17. A method for identifying compounds that interfere with HIV capsid assembly by specifically binding to and preventing said conformer from binding to HIV Gag, said method comprising the steps of:
a) expressing HIV Gag in a mammalian cell;
b) identification of co-localization of HIV Gag and HP68 using immunofluorescence in said mammalian cells, and;
c) screening said potential compounds for test compounds that interfere with co-localization of HP68 and Gag in said mammalian cells whereby compounds that interfer with HIV capsid assembly are identified by a diffuse staining pattern of HP68.

18. The method according to the method of claim 17, wherein said compounds do not cause toxcicity or upregulate host stress proteins in said mammalian cells.

19. A method of identifying a binding site between a host chaperone protein and an intermediate in HIV capsid assembly, said method comprising the steps of:
obtaining a conformational epitope map of said host protein using monoclonal antibodies produced according to the method claim 12;
obtaining a conformational epitope map of HIV Vif using monoclonal antibodies specific for Vif;
obtaining a conformational epitope map of HIV capsid intermediates using monoclonal antibodies specific for HIV Vif and antibodies produced according to the method of claim 12; and
comparing conformational epitope maps of HIV Vif, capsid intermediate complex and said conformer, whereby the binding site on said conformer for HIV Vif is identified and the binding site on HIV Vif for said conformer is isdentified.

20. A method for identifying compounds that interfere with HIV capsid assembly by specifically binding to and preventing said conformer from binding to HIV Vif, said method comprising the steps of:
(a) screening databases for compounds that bind to the binding site identified according to the method of claim 15, whereby potential compounds are obtained;
(c) screening said potential compounds for test compounds that bind substantially specifically to a host chaperone protein for HIV capsid assembly but not to conformers of said host chaperone proteins that do not bind HIV Vif,
(d) screening said test compounds in a cell free translation system, wherein efficacy is measured by a decrease in HIV capsid production; and
(g) further screening said compounds for ability to block HIV capsid formation in mammalian cells infected with HIV, whereby compounds that block HIV capsid formation are identified.

21. The method according to the method of claim 20, wherein said compounds do not cause toxcicity or upregulate host stress proteins in said mammalian cells.

22. A method for establishing a profile of host protein HIV capsid assembly chaperones and their conformers in a population of individuals infected with HIV, wherein said profile is relative to specific HIV characteristics, said method comprising the steps of:
compiling a conformer profile of host protein HIV capsid assembly chaperones and their conformers in individual members of said population, wherein said individual members produce HIV virions; and
establishing a relationship between said conformer profiles of said individual members and specific characteristics of HIV in said individual members, whereby a population profile of conformers relative to specific HIV characteristics is obtained.

23. A method for selecting a treatment to administer to a individual infected with HIV, said method comprising the steps of:
determining a conformer profile of host protein HIV capsid assembly chaperones and their conformers of said individual;
comparing said conformer profile of said patient to a conformer population profile obtained according to the method of claim 22; and
selecting as a method of treatment for said individual a method of treatment that was successful for treatment of individual members of said population having a substantially similar conformer profile, whereby a treatment based on a conformer profile is selected for said individual.

24. HIV capsid intermediates produced by a cell free system comprising:
a) HIV Gag Pr55 mRNA;
b) cell-free extract, amino acids, transfer RNA (tRNA), ribosomes and an energy source;
c) a concentration of myristoyl coenzyme A about 0.1 to 100 micromolar;
wherein said capsid intermediates are selected from the group consisting of proteins having a buoyant density of about 10S, about 80S, about 150S and about 500S.

25. The HIV capsid intermediates produced according to claim 24, wherein said intermediates comprise HIV capsid proteins and host chaperone proteins.

26. The HIV capsid intermediates produced according to claim 24, wherein said intermediates comprise Gag and HP68.

27. The HIV capsid intermediates produced according to claim 24, wherein said intermediates comprise Vif and HP68.

28. The HIV capsid intermediates produced according to claim 24, wherein said intermediates comprise HP68 that binds to Gag and Vif but does not bind to RNase L.

29. A cell-free system for translation and assembly of an HIV capsid, comprising a cell-free translation mixture, an mRNA molecule encoding a Gag Pr55 protein derived from human immunodeficiency virus (HIV), and myristoyl coenzyme A.

30. The cell-free translation system of claim 29, which further includes a detergent-sensitive fraction derived from eukaryotic cell membranes.

31. The cell-free translation system of claim 29, which further includes a eukaryotic cell component characterized by insensitivity to a concentration of at least 0.5% (wt/vol) “NIKKOL” detergent.

32. The cell-free translation system of claim 29, wherein said system further includes HIV genomic RNA or a fragment thereof.

33. The cell-free translation system of claim 29, which further includes (i) a DNA molecule which encodes HIV Gag Pr55, (ii) an RNA polymerase for synthesizing said mRNA, and (iii) sufficient concentrations of nucleotides ATP, UTP, GTP, and CTP to support such mRNA synthesis.

34. The cell-free translation system of claim 29, wherein said HIV Gag mRNA encodes a mutant defective in assembly.

35. A method of producing an HIV capsid intermediate in a cell-free system, comprising adding to a cell-free protein translation mixture which contains a cell-free extract, amino acids, transfer RNA (tRNA), ribosomes and an energy source: (i) an mRNA molecule encoding an HIV Pr55 Gag protein, and (ii) a concentration of myristoyl coenzyme A that is greater than about 0.1 micromolar, to form a reaction mixture;
incubating said reaction mixture for a period of time sufficient to assemble Gag Pr55 mRNA translation products into an immature HIV capsid.

36. The method of claim 35, wherein said reaction mixture is supplemented with (iii) a detergent-sensitive fraction derived from eukaryotic cell membranes, and (iv) a eukaryotic cell component characterized by insensitivity to a concentration of at least 0.5% (wt/vol) “NIKKOL” detergent.

37. The method of claim 35, wherein said reaction mixture is supplemented with host protein HP68 or a homolog thereof

38. The method of claim 35, which further includes adding to said reaction mixture an HIV genomic RNA molecule or a fragment thereof

39. The method of claim 35, which further includes adding to said reaction mixture a Gag
Pr55 DNA transcript and a transcription mixture containing an RNA polymerase and ribonucleotides ATP, UTP, GTP and CTP effective to produce said Gag mRNA in said cell-free mixture.

40. An isolated HIV capsid intermediate selected from a group of HIV capsid intermediates having buoyant densities selected from the group of about 10S, about 80S, about 150S and about 500S, wherein said buoyant densities are measured by sedimentation in a linear sucrose density gradient ranging from 15% to 60% sucrose.

41. A method of selecting a compound capable of altering HIV capsid assembly, comprising
adding a test compound to a reaction mixture which includes (i) a cell-free translation mixture that includes a cell-free extract, tRNA, ribosomes, amino acids and an energy source, (ii) an mRNA molecule encoding HIV Gag Pr55, (iii) myristoyl coenzyme A, present at a concentration greater than about 0.1 micromolar,
measuring capsid assembly in the presence of said test compound, comparing assembly in the absence of said test compound to assembly in the presence of said compound,
selecting the compound as a compound capable of altering HTV capsid assembly if assembly measured in the absence of said compound is significantly different than assembly measured in the presence of said compound.

42. The method of claim 41, wherein said measuring of capsid assembly includes measuring formation of assembly intermediates.

43. A host cell protein, comprising:
a peptide region having the sequence presented as SEQ ID NO: 2, specific immunoreactivity with monoclonal antibody 23 c, and an apparent molecular weight of about 68 kilodaltons,
wherein said protein associates with HIV capsid intermediates produced by the cell-free translation system of claim 29.

44. The host cell protein of claim 43, wherein said protein is characterized by at least 75% amino acid sequence identity to HP 68.

45. The host cell protein of claim 43, which is derived from wheat germ extract and which is identified as HP68.

46. A method of inhibiting HIV capsid formation in a cell, comprising adding to the cell a compound selected for its ability to inhibit HIV capsid formation in a cell-free translation system consisting essentially of (i) a cell-free translation mixture which contains a cell-free extract, tRNA, ribosomes, amino acids and an energy source, (ii) an mRNA molecule encoding a HIV capsid assembly protein Pr55, and (iii) myristoyl coenzyme A.

47. The method of claim 46, wherein said compound is selected for its ability to block association of the host protein of claim 41 or sequence homologs thereof with an HIV capsid intermediate.

48. A method of selecting a compound capable of altering HIV capsid assembly in cells, comprising
adding a test compound to cells that are forming retroviral capsids,
measuring the quantity and nature of capsid assembly intermediates formed within cells in the presence of said test compound,
comparing the quantity and nature of assembly intermediates formed within cells in the absence of said test compound to said quantity and nature of intermediates formed in the presence of test compound,
selecting the compound as a compound capable of altering formation of HIV assembly intermediates if the quantity or nature of intermediates measured in the presence of said compound is significantly different than the quantity or nature of intermediates measured in the absence of said compound.

49. The method of claim 48, wherein said selected retrovirus is HIV, and said measuring of said capsid formation is accomplished by measuring association of HP68 or a homolog thereof with an HIV capsid intermediate.

50. A method for encapsidating genomic HIV RNA or fragments thereof, comprising adding said RNA or RNA fragments to a cell-free translation system as defined in claim 29, and
incubating said system for a period of time sufficient to complete said translation and assembly reaction.
